(19) United States
(12) Patent Application Publication
(10) Pub. No.: US 2025/0018150 A1
(43) Pub. Date: Jan. 16, 2025

(54) APPARATUS FOR BENDING MALLEABLE MEMBER OF MEDICAL INSTRUMENT

(71) Applicants: Acclarent, Inc., Irvine, CA (US); Biosense Webster (Israel) Ltd., Yokneam (IL)

(72) Inventors: Brittanie Chu, Chino Hills, CA (US); Raymond Yue-sing Tang, Rosemead, CA (US); Alex Gonzalez, Lake Forest, CA (US)

(21) Appl. No.: 18/770,296

(22) Filed: Jul. 11, 2024

Related U.S. Application Data
(60) Provisional application No. 63/526,343, filed on Jul. 12, 2023.
(60) Provisional application No. 63/538,094, filed on Sep. 13, 2023.
(60) Provisional application No. 63/564,524, filed on Mar. 13, 2024.

(51) Int. Cl. A61M 25/00; A61F 11/20; A61M 29/02

(52) U.S. Cl. CPC A61M 25/0068; A61F 11/202; A61M 29/02; A61M 2205/3327; A61M 2209/04

(57) ABSTRACT

An apparatus includes a base, an actuator, and a clamp member. The actuator is pivotably coupled with the base. The clamp member is operable to selectively clamp a malleable guide rail relative to the base. The guide rail is configured to fit in an anatomical passageway in a head of a patient. The actuator is operable to pivot relative to the base to thereby bend the guide rail while the guide rail is clamped by the clamp member.

The present application claims priority under 35 U.S.C. § 119 to U.S. Patent Application Ser. No. 63/526,343, filed Jul. 12, 2023 and having the title “APPARATUS FOR BENDING MALLEABLE MEMBER OF MEDICAL INSTRUMENT,” to U.S. Patent Application Ser. No. 63/538,094, filed Sep. 13, 2023 and having the title “APPARATUS FOR BENDING MALLEABLE MEMBER OF MEDICAL INSTRUMENT,” and to U.S. Patent Application Ser. No. 63/564,524, filed Mar. 13, 2024 and having the title “APPARATUS FOR BENDING MALLEABLE MEMBER OF MEDICAL INSTRUMENT”—each of which is incorporated herein by reference in its entirety.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pat. No. 11,534,192, entitled “Methods and Apparatus for Treating Disorders of the Sinuses,” issued Dec. 27, 2022, the disclosure of which is incorporated by reference herein, in its entirety; U.S. Pat. No. 9,579,448, entitled “Balloon Dilation Catheter System for Treatment and Irrigation of the Sinuses,” issued Feb. 28, 2017, the disclosure of which is incorporated by reference herein, in its entirety; U.S. Pat. No. 9,155,492, entitled “Sinus Illumination Lightwire Device,” issued Oct. 13, 2015, the disclosure of which is incorporated by reference herein, in its entirety; and U.S. Pub. No. 2021/0361912, entitled “Shaft Deflection Control Assembly for ENT Guide Instrument,” published Nov. 25, 2021, the disclosure of which is incorporated by reference herein, in its entirety.

In the context of Eustachian tube dilation, a dilation catheter or other dilation instrument may be inserted into the Eustachian tube and then be inflated or otherwise expanded to thereby dilate the Eustachian tube. The dilated Eustachian tube may provide improved ventilation from the nasopharynx to the middle ear and further provide improved drainage from the middle ear to the nasopharynx. Methods and devices for dilating the Eustachian tube are disclosed in U.S. Pat. No. 10,206,821, entitled “Eustachian Tube Dilation Balloon with Ventilation Path,” issued Feb. 19, 2019, the disclosure of which is incorporated by reference herein, in its entirety; and U.S. Pat. No. 11,013,896, entitled “Method and System for Eustachian Tube Dilation,” issued May 25, 2021, the disclosure of which is incorporated by reference herein, in its entirety.

Some medical instruments may include an adjustable guide that allows the same medical instrument to readily access different anatomical structures (e.g., Eustachian tubes and different passageways associated with drainage of paranasal sinuses, etc.). Examples of dilation instruments with adjustable guides are described in U.S. Pat. No. 10,137,285, entitled “Balloon Dilation System with Malleable Internal Guide,” issued Nov. 27, 2018, the disclosure of which is incorporated by reference herein, in its entirety; U.S. Pat. No. 11,013,897, entitled “Apparatus for Bending Malleable Guide of Surgical Instrument,” issued May 25, 2021, the disclosure of which is incorporated by reference herein, in its entirety; and U.S. Pat. No. 11,534,192, entitled “Methods and Apparatus for Treating Disorders of the Sinuses,” issued Dec. 27, 2022, the disclosure of which is incorporated by reference herein, in its entirety.

In some scenarios, it may be desirable to allow a dilation catheter of a medical instrument to translate longitudinally relative to a guide of the same instrument. This may allow the guide to be initially positioned in relation to a targeted anatomical passageway while the dilation catheter is in a proximal position. The dilation catheter may then be advanced relative to the guide to a distal position to thereby enter the targeted anatomical passageway. While several systems and methods have been made and used to dilate anatomical passageways within a patient, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a perspective view of an example of a bending tool;

FIG. 2 depicts a side elevation view of an actuator of the bending tool of FIG. 1;

FIG. 3 depicts a perspective view of a clamp member of the bending tool of FIG. 1;

FIG. 4 depicts a perspective view of a base of the bending tool of FIG. 1;

FIG. 5 depicts a cross-sectional view of the base of FIG. 4, taken along line 5-5 of FIG. 4;

FIG. 6A depicts a top plan view of the bending tool of FIG. 1 in a first stage of operation;

FIG. 6B depicts a top plan view of the bending tool of FIG. 1 in a second stage of operation;

FIG. 6C depicts a top plan view of the bending tool of FIG. 1 in a third stage of operation;

FIG. 6D depicts a top plan view of the bending tool of FIG. 1 in a fourth stage of operation;

FIG. 6E depicts a top plan view of the bending tool of FIG. 1 in a fifth stage of operation;

FIG. 6F depicts a top plan view of the bending tool of FIG. 1 in a sixth stage of operation;

FIG. 6G depicts a top plan view of the distal portion of a shaft assembly after having a guide rail bent by the bending tool of FIG. 1;

FIG. 7A depicts a top plan view of the shaft assembly of FIG. 6F with the bent guide rail inserted into a straightening feature of the bending tool of FIG. 1;

FIG. 7B depicts a top plan view of the shaft assembly of FIG. 6F and the bending tool of FIG. 1, with straightening feature of the bending tool of FIG. 1 having returned the bent guide rail to a straight configuration;

FIG. 8 depicts a perspective view of another example of a bending tool;

FIG. 9 depicts another perspective view of the bending tool of FIG. 8;

FIG. 10 depicts an exploded perspective view of the bending tool of FIG. 8;

FIG. 11 depicts a top cross-sectional view of the bending tool of FIG. 8, taken along line 11-11 of FIG. 8;

FIG. 12 depicts a top cross-sectional view of the bending tool of FIG. 8, taken along line 12-12 of FIG. 8;

FIG. 13A depicts a top plan view of the bending tool of FIG. 8 in a first stage of operation;

FIG. 13B depicts a top plan view of the bending tool of FIG. 8 in a second stage of operation;

FIG. 14 depicts a top plan view of the bending tool of FIG. 8 rotated 180 degrees to transition from a first mode of operation to a second mode of operation;

FIG. 15 depicts a perspective view of another example of a bending tool;

FIG. 16 depicts a top plan view of the bending tool of FIG. 15;

FIG. 17 depicts a perspective view of another example of a bending tool;

FIG. 18 depicts a top plan view of the bending tool of FIG. 15;

FIG. 19 depicts a perspective view of another example of a clamp member that may be incorporated into the bending tool of FIG. 1;

FIG. 20 depicts a cross-sectional view of the bending tool of FIG. 1 with the clamp member of FIG. 19;

FIG. 21 depicts a perspective view of another example of a bending tool;

FIG. 22 depicts a side elevation view of an actuator of the bending tool of FIG. 21;

FIG. 23 depicts a perspective view of a clamp member of the bending tool of FIG. 21;

FIG. 24 depicts a perspective view of a base of the bending tool of FIG. 21;

FIG. 25 depicts a cross-sectional view of the base of FIG. 24;

FIG. 26A depicts a top plan view of the bending tool of FIG. 21 in a first stage of operation;

FIG. 26B depicts a top plan view of the bending tool of FIG. 21 in a second stage of operation;

FIG. 26C depicts a top plan view of the bending tool of FIG. 21 in a third stage of operation;

FIG. 26D depicts a top plan view of the bending tool of FIG. 21 in a fourth stage of operation;

FIG. 26E depicts a top plan view of the bending tool of FIG. 21 in a fifth stage of operation;

FIG. 26F depicts a top plan view of the bending tool of FIG. 21 in a sixth stage of operation;

FIG. 26G depicts a top plan view of the distal portion of a shaft assembly after having a guide rail bent by the bending tool of FIG. 21; and

FIG. 27A depicts a top plan view of the shaft assembly of FIG. 26F with the bent guide rail inserted into a straightening feature of the bending tool of FIG. 1; and

FIG. 27B depicts a top plan view of the shaft assembly of FIG. 26F and the bending tool of FIG. 21, with straightening feature of the bending tool of FIG. 1 having returned the bent guide rail to a straight configuration.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of Bending Tool

In some scenarios, it may be desirable to advance a dilation catheter into an anatomical passageway in or near the ear, nose, or throat of a patient; and expand the dilator to thereby dilate the passageway. For instance, it may be desirable to dilate a paranasal sinus ostium or other passageway associated with drainage of a paranasal sinus cavity, a Eustachian tube, a stenotic region in an airway of a patient, etc. It may also be desirable to incorporate a guide into such an instrument, to assist in guiding the dilation catheter into the targeted anatomical passageway; and to allow the dilation catheter to translate longitudinally relative to the guide. This may allow the guide to be initially positioned in relation to a targeted anatomical passageway while the dilation catheter is in a proximal position. The dilation catheter may then be advanced relative to the guide to a distal position to thereby enter the targeted anatomical passageway.

In some scenarios, it may be desirable for the guide of a dilation instrument to have a malleable portion that allows the operator to bend the guide to achieve different bend angles. The malleability of the guide may allow the guide to be bent to a desired bend angle before being inserted into the head of the patient. The malleability of the guide may allow the guide to maintain the bend angle of the bend while the guide is disposed in the head of the patient, including while a dilation catheter is advanced distally relative to the guide. Such operability of the guide may promote access by a dilation catheter to various locations within the head of a patient, such as the maxillary sinus ostium, the frontal recess, the sphenoid sinus ostium, the Eustachian tube, etc., based on the selected bend angle.

In scenarios where a dilation instrument has a malleable guide element, it may be desirable to provide a tool that is operable to facilitate bending of the malleable guide element to achieve a desired bend angle with a suitable degree of precision. By way of example only, the bending of a guide of a dilation instrument may be performed in accordance with at least some of the teachings of U.S. Pat. No. 11,013,897, entitled “Apparatus for Bending Malleable Guide of Surgical Instrument,” issued May 25, 2021, the disclosure of which is incorporated by reference herein, in its entirety. In addition to providing a suitable degree of precision in the angular extent of the bend angle, it may also be desirable to facilitate formation of the bend angle at a desired position along the length of the malleable guide element. Moreover, it may be desirable to facilitate straightening of the malleable guide element after the malleable guide element has been bent. The following description provides examples of a bending tool that is operable to provide a selected bend angle at a selected position along the length of a malleable guide element; and to facilitate straightening of the malleable guide element after the malleable guide element has been bent.

### A. First Example of Bending Tool

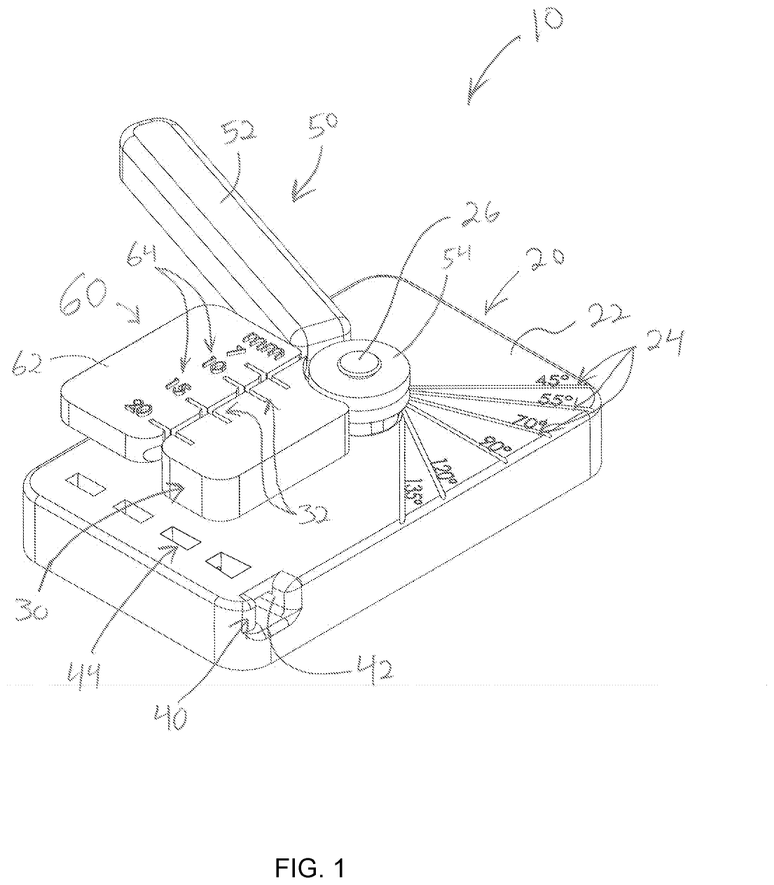

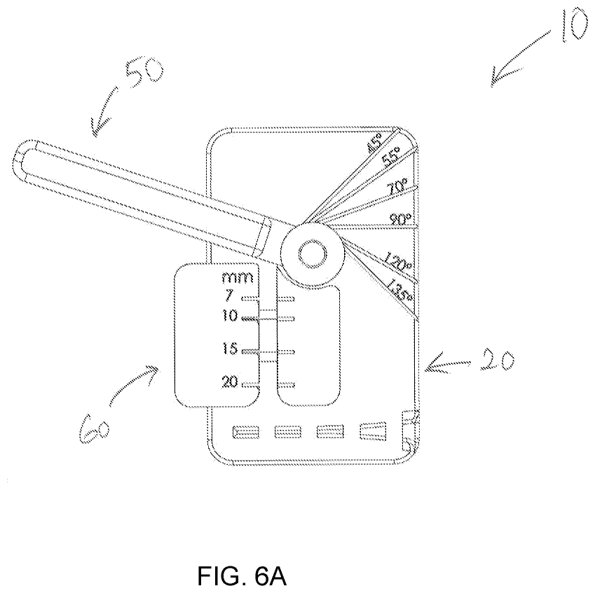

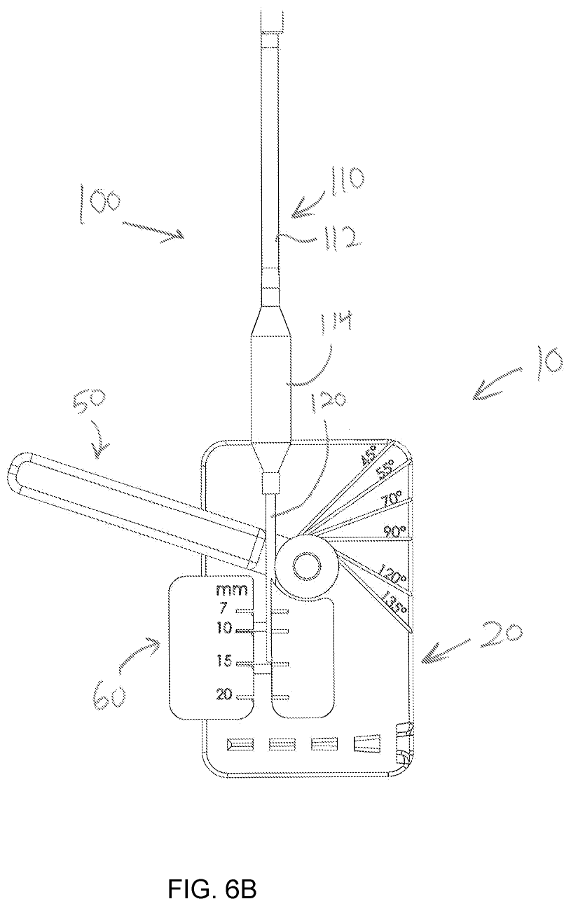

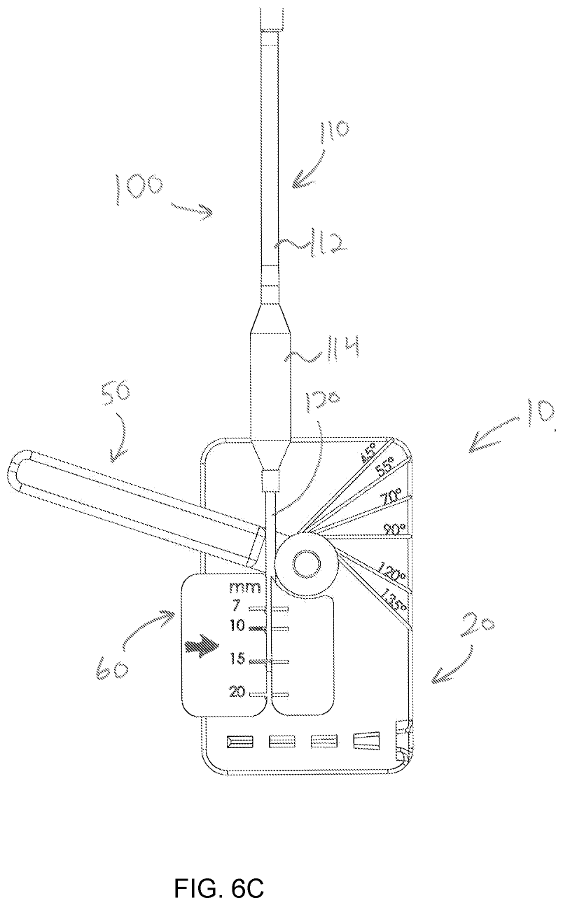

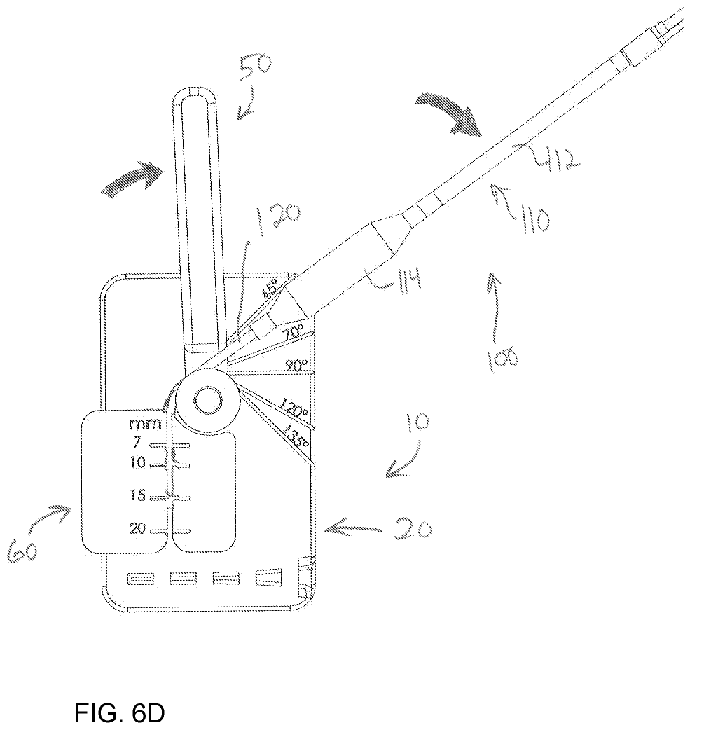

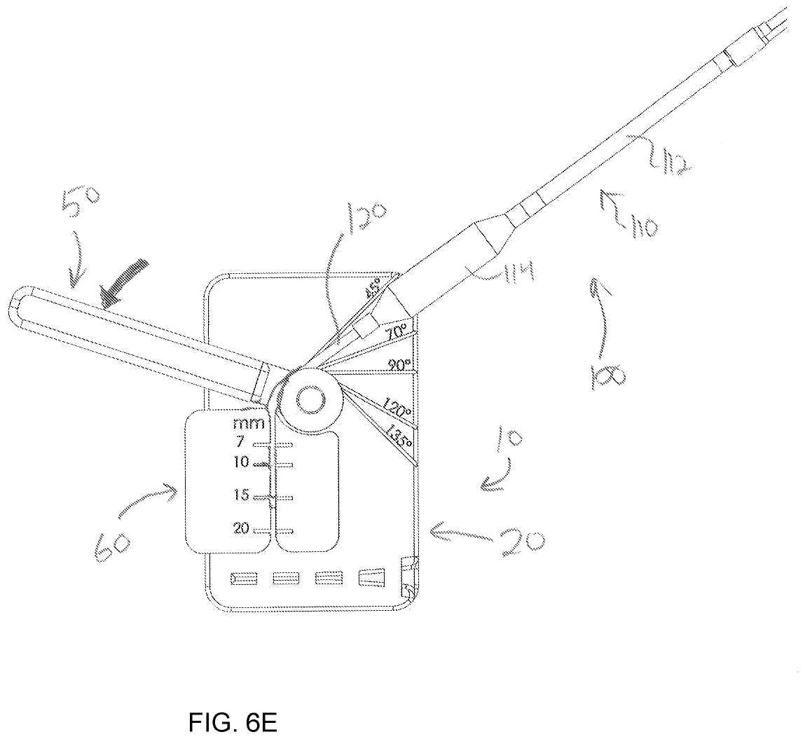

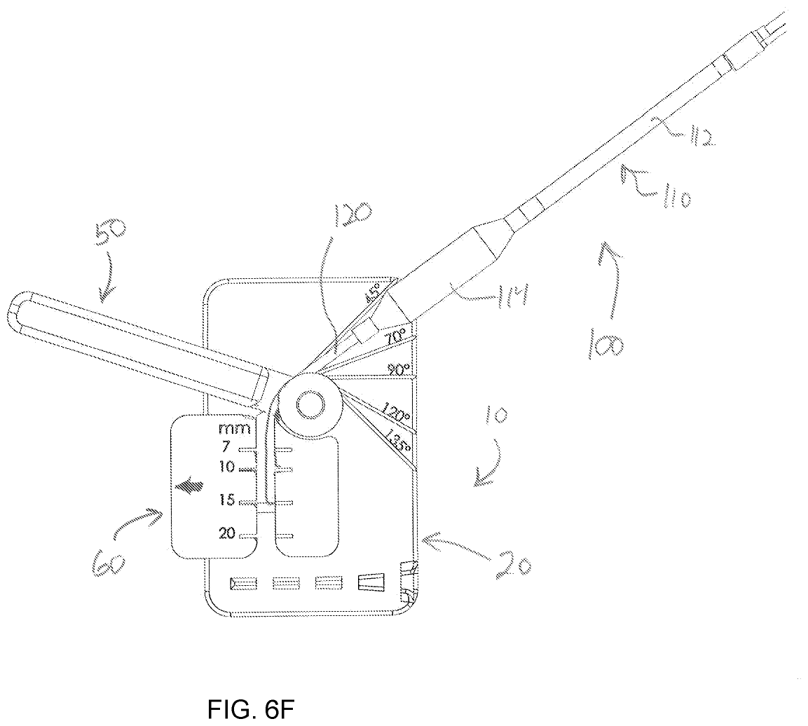

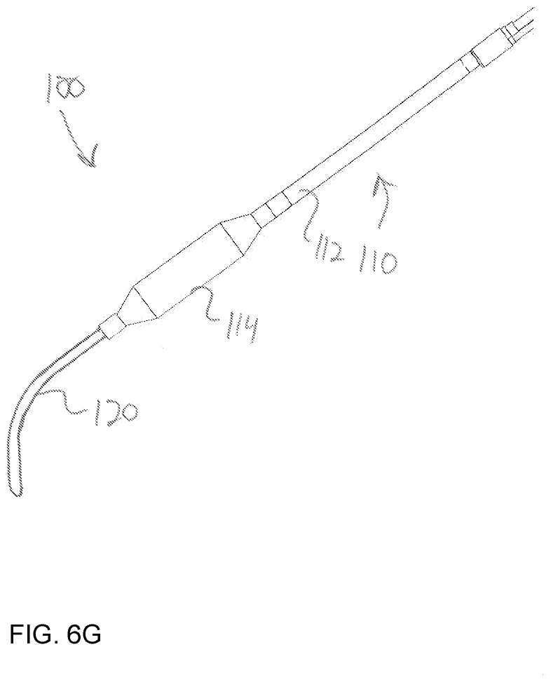

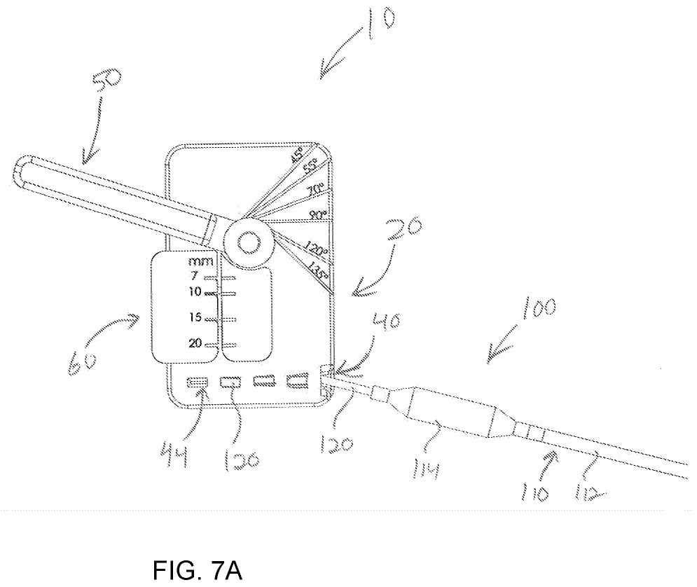

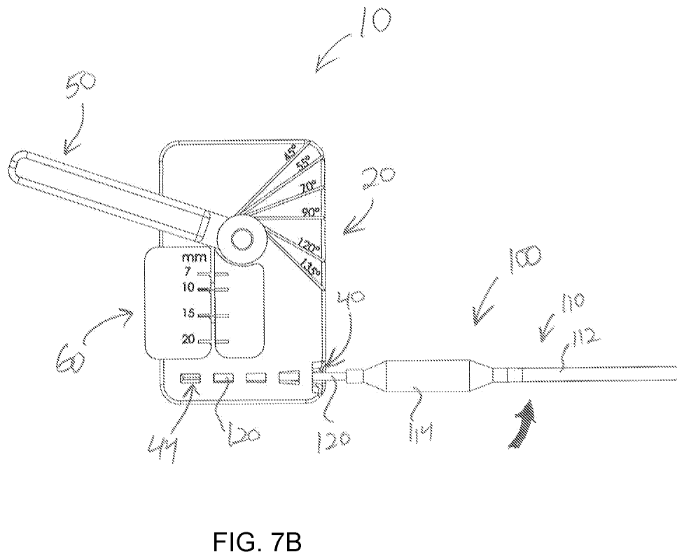

FIG. 1 shows an example of a bending tool 10 that may be used to bend a guide rail 120 of a shaft assembly 100. As shown in FIGS. 6B-7B, the shaft assembly 100 of this example includes a dilation catheter 110 having a shaft 112 and a balloon 114, which are slidably disposed along a guide rail 120. The distal portion of the shaft assembly 100 is configured to fit in an anatomical passageway within the head of a patient, such as the maxillary sinus ostium, the frontal recess, the sphenoid sinus ostium, the Eustachian tube, etc. The balloon 114 is configured to expand within the anatomical passageway to thereby dilate the anatomical passageway and/or provide other effects on/within the anatomical passageway. The guide rail 120 comprises a malleable material that is configured to allow an operator to bend the guide rail 120 to a desired bend angle. Moreover, the guide rail 120 will maintain the operator-induced bend angle as the guide rail 120 is inserted into or near the targeted anatomical passageway; and as the dilation catheter 110 is advanced distally along the guide rail to position the balloon 114 within the targeted anatomical passageway. The shaft assembly 100 may be configured and operable like any of the shaft assemblies of the dilation instruments described in any of the various references cited herein. While the shaft assembly 100 is used in the clinical context of ear, nose, and throat procedures in the present example, it should be understood that the bending tool 10 may be used with various kinds of instruments that are used in various kinds of clinical contexts.

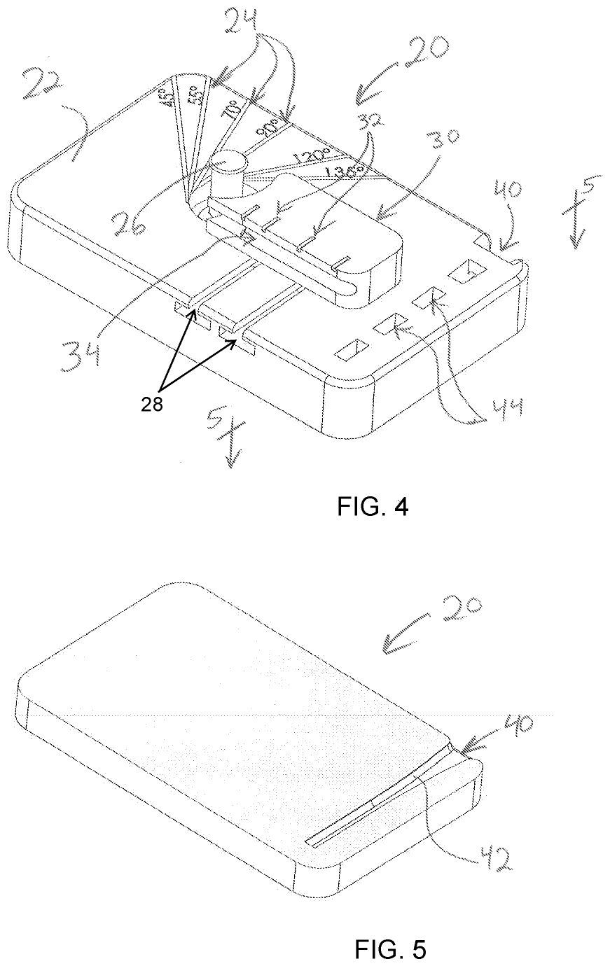

As shown in FIG. 1, the bending tool 10 of the present example comprises a base 20, an actuator 50, and a clamp member 60. As best seen in FIGS. 1 and 4, the base 20 includes an upper surface 22 with a series of indicia 24, an upwardly extending post 26, an upwardly extending block 30, and a laterally extending recess 40. The indicia 24 are configured to visually indicate various bend angles as will be described in greater detail below. The post 26 is configured to provide an axle about which the actuator 26 pivots relative to the base 20, as will also be described in greater detail below. The block 30 is configured to cooperate with the clamp member 60 to secure a distal portion of the guide rail 120 relative to the base 20, as will also be described in greater detail below. The block 30 includes a set of windows 32 and a radiused recess 34. The windows 32 are configured to provide indicia as described in greater detail below. The recess 34 also has a radius that is configured to complement the radius of the guide rail 120, such that a laterally facing region of the guide rail 120 may fit in the recess 34 during operation of the bending tool 10 as described in greater detail below.

As best seen in FIG. 5, the recess 40 extends laterally into the side of the base 20, without passing fully through the base 20 in this version; and has a tapered surface 42 such that the opening of the recess 40 is wider than the furthest interior region of the recess 40. In the present example, the taper is curved, though in other versions, the taper may be angled or otherwise configured. In some other versions, the recess 40 passes fully through the entre width of the base 20. In some such versions, each open end of the recess 40 is wider than the interior region of the recess 40.

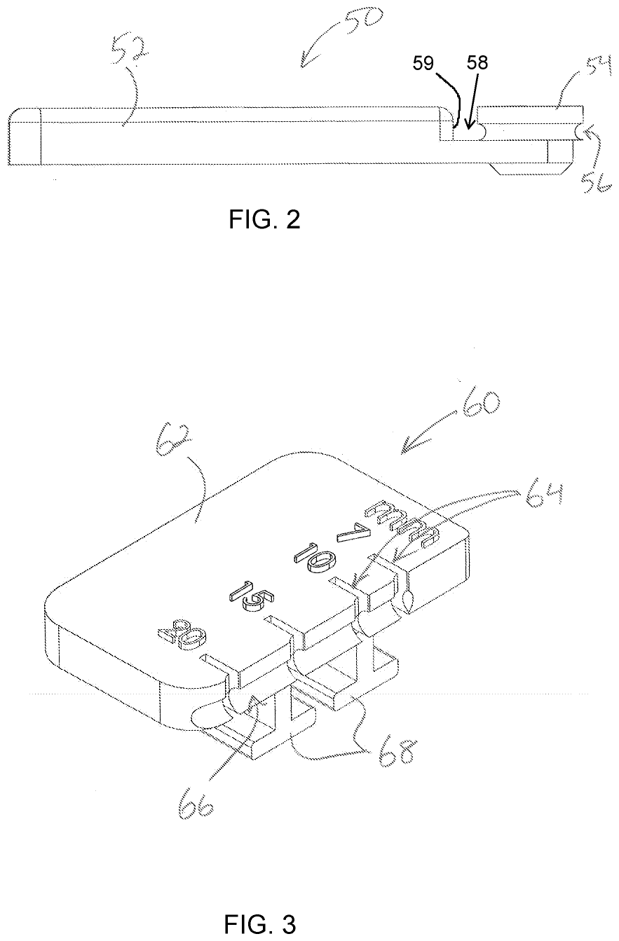

As shown in FIGS. 1-2, the actuator 50 comprises a lever arm 52 and a hub 54. The hub 54 is pivotably secured to the base 20 via the post 26, such that the lever arm 52 is pivotable about a central axis defined by the post 26. As best seen in FIG. 2, the actuator 50 further includes a radiused annular recess 56 in the hub 54. In the present example, the annular recess 56 has a radius that is configured to complement the radius of the guide rail 120, such that a laterally facing region of the guide rail 120 may fit in the annular recess 56 during operation of the bending tool 10 as described in greater detail below. As also best seen in FIG. 2, a gap 58 is defined between the annular recess 56 and a bearing surface 59 of the lever arm 52.

As shown in FIGS. 1 and 3, the clamp member 60 comprises a body 62 having a set of windows 64, a radiused recess 66, and a pair of rails 68. The windows 64 are configured to correspond with the windows 32 and provide indicia as described in greater detail below. The rails 68 are configured to fit in the complementary recesses 28 formed in the base 20, such that the clamp member 60 is slidably supported by the base 20. In the present example, the rails 68 and the recesses 28 have complementary T-shapes, though the rails 68 and the recesses 28 may alternatively have any other suitable shapes. The recess 66 has a radius that is configured to complement the radius of the guide rail 120, such that a laterally facing region of the guide rail 120 may fit in the recess 66 during operation of the bending tool 10 as described in greater detail below. The recess 66 of the clamp member 60 faces the recess 34 of the block 30, such that the guide rail 120 may be captured within the recesses 34, 66 during operation of the bending tool 10. While each of the recesses 34, 66 has a semicircular, radiused profile in this example, either or both of the recesses 34, 66 may have any other suitable kind of profile, including but not limited to a V-shaped profile. In some variations, one of the recesses 34, 66 is omitted. Some variations of the bending tool 10 may also include detent features that provide tactile feedback and/or resistance against inadvertent movement of the clamp member 60 relative to the block 30. For instance, such detent features may provide tactile feedback and/or resistance against inadvertent movement of the clamp member 60 relative to the block 30 when the clamp member 60 is in a fully open position (e.g., as shown in FIGS. 6A-6B and 6F) and/or when the clamp member 60 is in a closed position (e.g., as shown in FIGS. 6C-6E).

As shown in FIG. 6A, operation of the bending tool 10 may begin with the actuator 50 pivoted fully counterclockwise; and with the clamp member 60 slid to the left to maximize the gap between the clamp member 60 and the block 30. Next, as shown in FIG. 6B, the shaft assembly 100 may be positioned relative to the bending tool 10 such that the guide rail 120 is disposed in the gap 58; and such that the guide rail 120 is positioned to a desired depth between the clamp member 60 and the block 30. The depth of insertion of the guide rail 120 into the space between the clamp member 60 and the block 30 will determine the position of the bend angle along the guide rail 120. To facilitate achieving the desired depth of insertion, the operator may visually observe the position of the distal tip of the guide rail 120 in relation to the windows 32, 64. As shown, the clamp member 60 may further include depth markings for each of the windows 64. It should also be understood that the block 30 may include depth markings for each of the windows 32, in addition to or as an alternative to the clamp member 60 including depth markings for each of the windows 64. The depth of insertion (and thus the longitudinal position of the bend along the length of the guide rail 120 may be chosen based on the targeted anatomical structure, based on the anatomy of the patient at hand, and/or based on other considerations.

Once the shaft assembly 100 has been positioned relative to the bending tool 10 to achieve the desired depth of insertion of the guide rail 120 into the space between the clamp member 60 and the block 30, the operator may slide the clamp member 60 toward the block 30, to thereby capture the distal portion of the guide rail 120 in the recesses 34, 66, as shown in FIG. 6C. The configuration of the windows 32, 64 may allow the operator to visually confirm that the distal tip of the guide rail 120 is at the desired position after the distal portion of the guide rail 120 is captured in the recesses 34, 66. The operator may maintain a firm grip on the base 20 and the clamp member 60, to thereby firmly clamp the distal portion of the guide rail 120 between clamp member 60 and the block 30. In some versions, the recesses 34, 66 include an elastomeric coating or overmold feature that further promotes gripping of the guide rail 120 between the clamp member 60 and the block 30.

With the distal portion of the guide rail 120 suitably clamped between the clamp member 60 and the block 30, the operator may then pivot the actuator 50 clockwise relative to the base 20 as shown in FIG. 6D. During this movement of the clamp member 60, the bearing surface 59 and the hub 54 may cooperate to form a bend in the guide rail 120. The operator may visually observe the guide rail 120 in relation to the indicia 24 to confirm when the desired bend angle has been achieved. After achieving the desired bend angle, the operator may pivot the actuator 50 counterclockwise relative to the base 20 as shown in FIG. 6E. The operator may then slide the clamp member 60 away from the block 30, to thereby release the distal portion of the guide rail 120, as shown in FIG. 6F. The operator may then remove the bending tool from the shaft assembly 100, leaving the guide rail 120 in a bent state with the formed bend having the desired bend angle at the desired longitudinal position along the length of the guide rail 120, as shown in FIG. 6G. After forming the bend with the desired bend angle at the desired longitudinal position along the length of the guide rail 120, the operator may use the shaft assembly 100 to dilate an anatomical passageway in the ear, nose, or throat of the patient; or otherwise use the shaft assembly 100.

In some cases, the operator may wish to remove the bend that was formed in the guide rail 120. To that end, the operator may insert the bent guide rail 120 into the recess 40 of the base 20 as shown in FIG. 7A. In the present example, the base 20 includes a set of windows 44 formed through the upper surface 22, allowing the operator to visually observe the depth of insertion of the guide rail 120 into the recess 40. In some other versions, the windows 44 are omitted. With the guide rail 120 suitably disposed in the recess 40, the operator may grasp the base 20 with one hand and manipulate the shaft assembly 100 with the other hand to thereby bend the guide rail 120 back to a straightened state as shown in FIG. 7B. In some instances, the operator may re-bend the guide rail 120 to a new bend angle, perhaps at a different position along the length of the guide rail 120, using the procedure described above with reference to FIGS. 6A-6G. These bending and straightening processes may be repeated any desired number of times. In some scenarios, these processes are repeated to allow the shaft assembly 100 to be used to perform dilations in two or more different anatomical passageways (e.g., the maxillary sinus ostium, the frontal recess, the sphenoid sinus ostium, the Eustachian tube, etc.).

### B. Second Example of Bending Tool

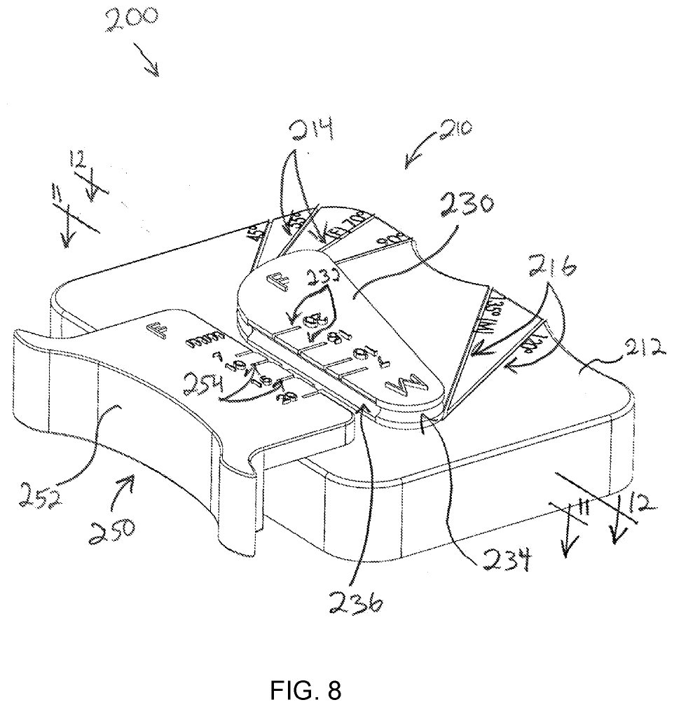

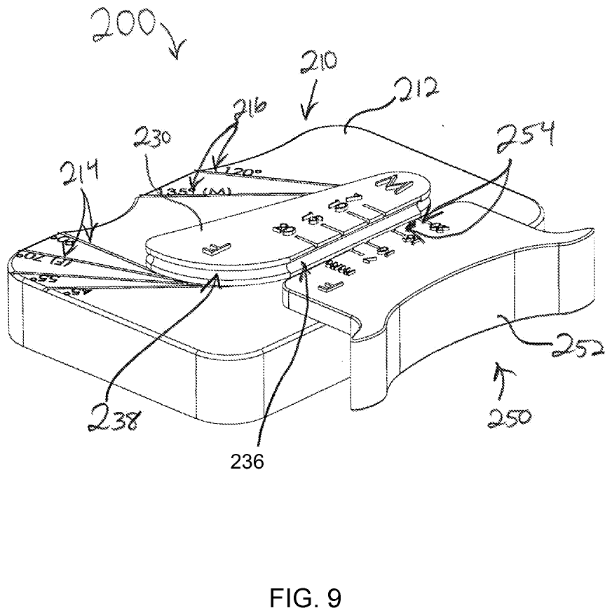

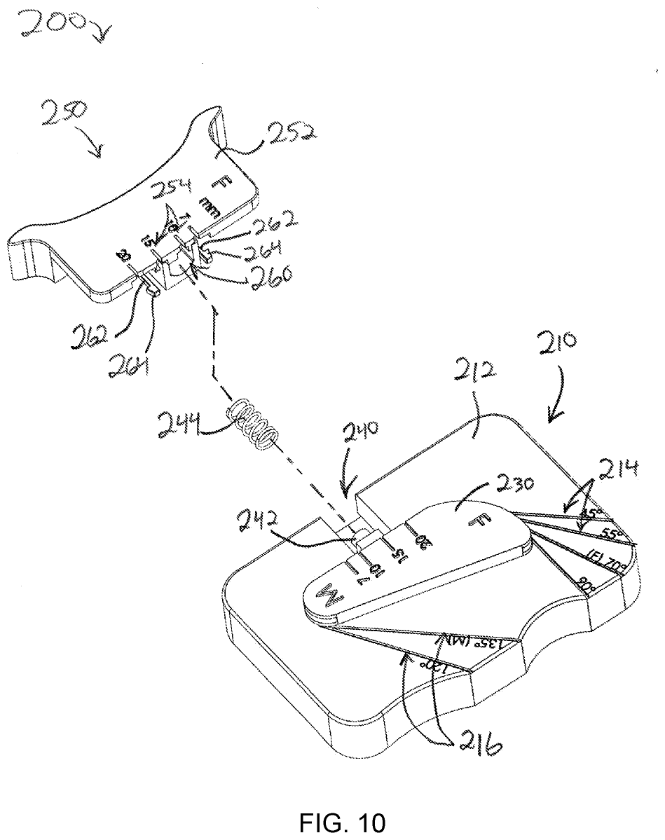

FIGS. 8-14 show another example of a bending tool 200 that may be used to bend a guide rail 120 of a shaft assembly 100. Except as otherwise described below, the bending tool 200 of this example may be configured and operable like the bending tool 10 described above. The bending tool 200 of this example includes a base 210 and a clamp member 250. As best seen in FIGS. 8-10 and 13A-14, the base 210 includes an upper surface 212 with a first set of indicia 214 and a second set of indicia 216, and an upwardly extending block 230. As best seen in FIGS. 10-12, the base further includes a laterally extending recess 240. A spring mount 242 and a pair of latch engaging shoulders 246 are positioned within recess 240.

The block 230 is configured to cooperate with the clamp member 250 to secure a distal portion of the guide rail 120 relative to the base 210. The block 230 includes a set of windows 232, a first radiused recess 234, a second radiused recess 236, and a third radiused recess 238. The windows 232 are configured to provide indicia as described in greater detail below. Each of the recesses 234, 236, 238 also has a radius along a first plane (e.g., vertical plane) that is configured to complement the radius of the guide rail 120, such that a laterally facing region of the guide rail 120 may fit in one or more of the recesses 234, 236, 238 during operation of the bending tool 200 as described in greater detail below. While each of the recesses 234, 236, 238 has the same radius of curvature extending inwardly into the block 230 along a first plane in this example, each of the recesses 234, 236, 238 is configured differently from the other recesses 234, 236, 238 around the perimeter of the block 230. In particular, the first recess 234 has a first radius of curvature along a second plane (e.g., horizontal plane), along a first region of the perimeter of the block 230. The second recess 236 extends along a straight path along a second region of the perimeter of the block 230, such that the second recess 236 does not have a radius of curvature along the second plane. The third recess 238 has a second radius of curvature along the second plane, along a third region of the perimeter of the block 230. The second radius of curvature is larger than the first radius of curvature. These differences in the radii of curvature of the recesses 234, 238 along the second plane may facilitate achievement of bend angles that are particularly suited for access by the guide rail 120 into different respective anatomical passageways, as described in greater detail below.

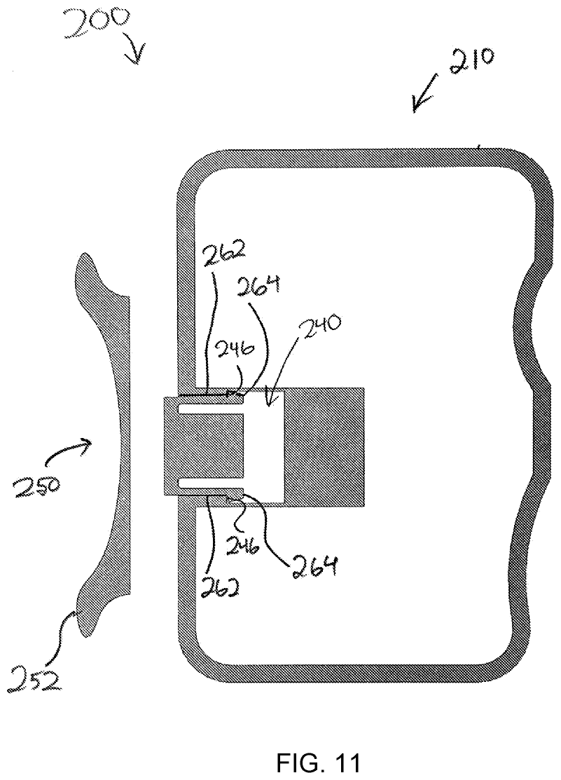

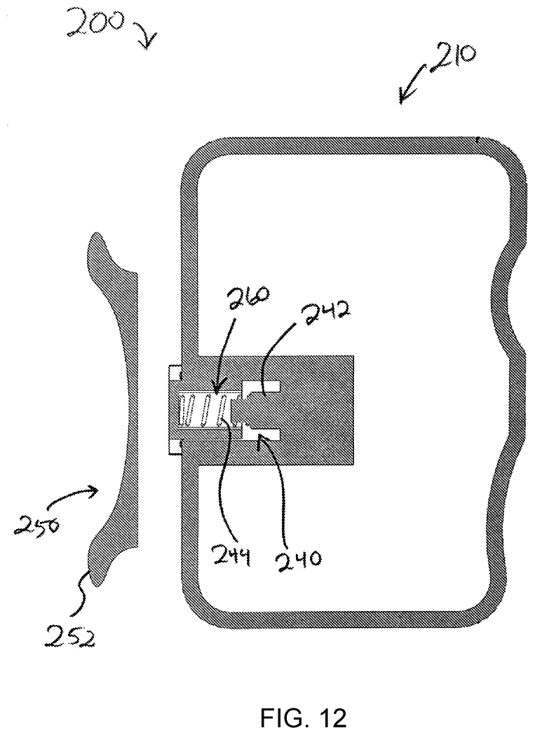

As shown in FIGS. 8-10 and 13A-14, the clamp member 250 comprises a body 252 having a set of windows 254, a recess 266, and a pair of resilient arms 262. The windows 254 are configured to provide indicia as described in greater detail below. The recess 260 is configured to receive a spring 244, which is also configured to seat against the spring mount 242 in the recess 240 of the base 210, as best seen in FIG. 12. The spring 244 is configured to resiliently urge the clamp member 250 away from the base 210, though the spring 244 is compressible to allow the clamp member 250 to be driven toward the base 210 as described in greater detail below. Each of the arms 262 of the present example has a latch head 264. Each of the latch heads 264 is configured to engage a respective latch engaging shoulder 246 in the recess 240 of the base 210, as best seen in FIG. 11. The latch heads 264 thus cooperate with the latch engaging shoulders 246 to restrict movement of the clamp member 250 away from the base 210, despite the resilient urging of the spring 244. During assembly of the bending tool 200, arms 262 may temporarily deform toward each other to provide a snap fit between the clamp member 264 and the base 210.

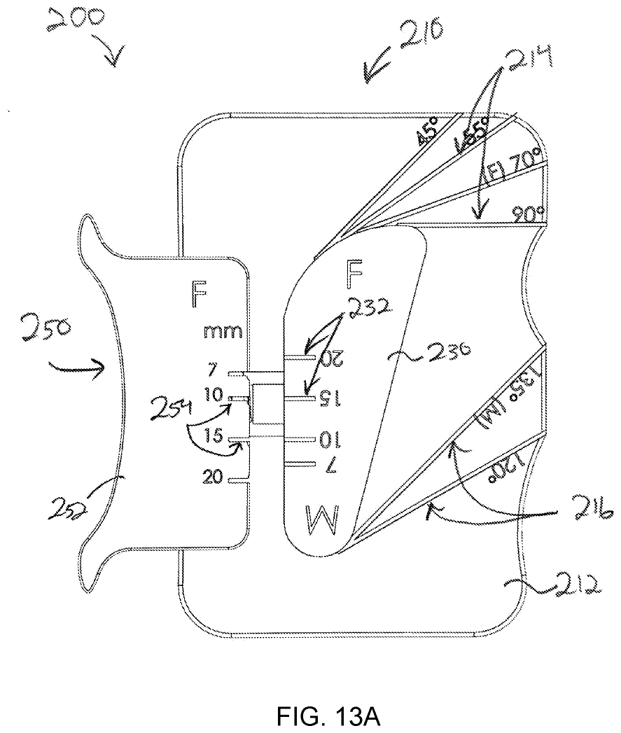

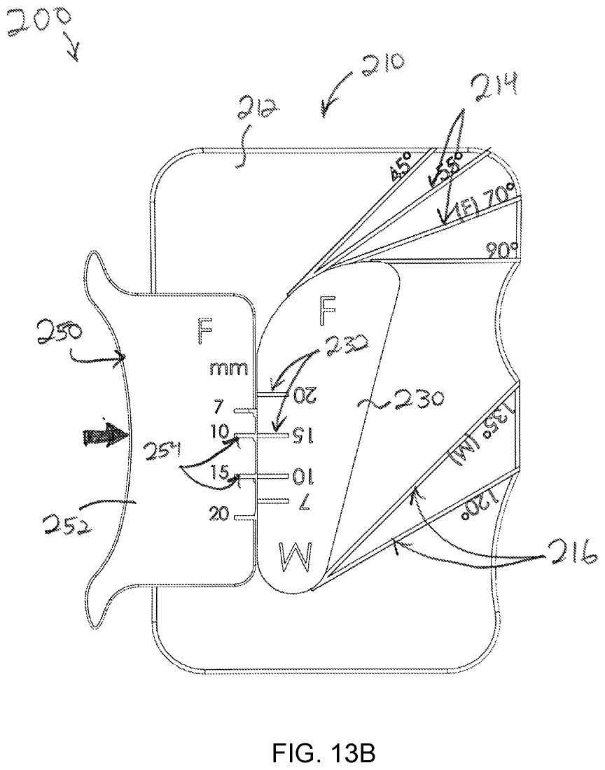

FIGS. 13A-13B show the bending tool 200 oriented for a first mode of operation. In this example, the first mode of operation is used to bend the guide rail 120 for insertion into a frontal recess of a patient. As shown, the letter “F” is positioned right-side-up on the clamp member 250 and on the block 230 to visually indicate to the operator that the bending tool 200 is oriented for the first mode of operation corresponding to the frontal recess. In addition, the depth-indicating numbers next to the windows 254 on the clamp member 250 are positioned right-side-up in this mode of operation, while the depth-indicating numbers next to the windows 232 on the block 230 are positioned upside-down. This relative orientation of the depth-indicating numbers next to the windows 254, 232 will further visually indicate to the operator that only the depth-indicating numbers next to the windows 254 should be viewed during this first mode of operation.

To operate the bending tool 200 in this first mode of operation, the operator would insert the guide rail 120 into the space defined between the clamp member 250 and the block 230 from the top (as viewed from the perspective of FIGS. 13A-13B). The operator would also view the position of the distal tip of the guide rail 120 in relation to the windows 254 to ensure that the guide rail 120 is inserted to the desired depth. The operator would then urge the clamp member 250 toward the block 230 as shown in the transition from the state shown in FIG. 13A to the state shown in FIG. 13B, thereby clamping the guide rail 120 against the block 230. With one hand grasping the bending tool 200 and the other hand grasping the instrument having the shaft assembly 100, while the guide rail 120 is clamped between the clamp member 250 and the block 230, the operator would bend the guide rail along the third radiused recess 238. The operator would continue this bending motion while observing the position of the shaft assembly 100 in relation to the indicia 214, until the shaft assembly 100 is aligned with the indicia 214 indicating the desired bend angle.

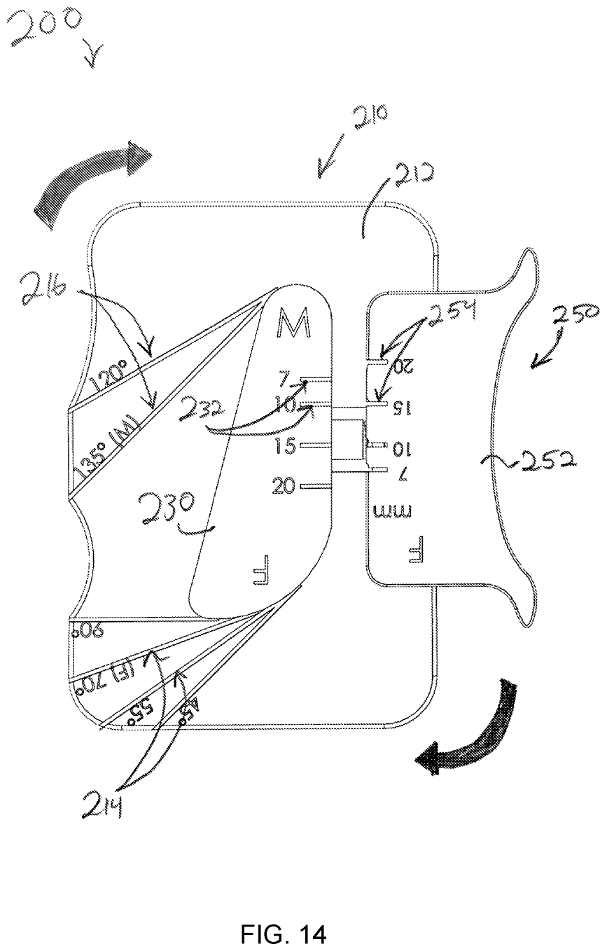

FIG. 14 shows the bending tool 200 oriented for a second mode of operation. In this example, the second mode of operation is used to bend the guide rail 120 for insertion into a maxillary sinus ostium of a patient. As shown, the letter “M” is positioned right-side-up on the clamp member 250 and on the block 230 to visually indicate to the operator that the bending tool 200 is oriented for the second mode of operation corresponding to the maxillary sinus. In addition, the depth-indicating numbers next to the windows 232 on the block 230 are positioned right-side-up in this mode of operation, while the depth-indicating numbers next to the windows 254 on the clamp member 250 are positioned upside-down. This relative orientation of the depth-indicating numbers next to the windows 254, 232 will further visually indicate to the operator that only the depth-indicating numbers next to the windows 232 should be viewed during this second mode of operation.

To operate the bending tool 200 in this second mode of operation, the operator would insert the guide rail 120 into the space defined between the clamp member 250 and the block 230 from the top (as viewed from the perspective of FIG. 14). The operator would also view the position of the distal tip of the guide rail 120 in relation to the windows 232 to ensure that the guide rail 120 is inserted to the desired depth. The operator would then urge the clamp member 250 toward the block 230 as described above with reference to the transition from the state shown in FIG. 13A to the state shown in FIG. 13B, thereby clamping the guide rail 120 against the block 230. With one hand grasping the bending tool 200 and the other hand grasping the instrument having the shaft assembly 100, while the guide rail 120 is clamped between the clamp member 250 and the block 230, the operator would bend the guide rail along the third radiused recess 234. The operator would continue this bending motion while observing the position of the shaft assembly 100 in relation to the indicia 216, until the shaft assembly 100 is aligned with the indicia 216 indicating the desired bend angle.

As noted above, the first recess 234 has a radius of curvature along a second plane (e.g., horizontal plane) along the perimeter of the block 230 that is smaller than the radius of curvature of the third recess 238 along the same plane along the perimeter of the block 230. This smaller radius of curvature of the first recess 234 along the second plane may facilitate achieving larger bend angles in the guide rail 120, such as those bend angles tailored to facilitate insertion of the guide rail 120 into a maxillary sinus ostium. By way of example only, these larger bend angles may range from approximately 120 degrees to approximately 135 degrees; or be otherwise greater than 90 degrees. By contrast, the larger radius of curvature of the third recess 238 along the second plane may facilitate achieving smaller bend angles in the guide rail 120, such as those bend angles tailored to facilitate insertion of the guide rail 120 into a frontal recess and/or other anatomical passageways. By way of example only, these smaller bend angles may range from approximately 45 degrees to approximately 90 degrees.

While the example described above correlates the first mode of operation and the third recess 238 with bending the guide rail 120 for insertion into the frontal recess, it should be understood that the first mode of operation and the third recess 238 may also be used to bend the guide rail 120 for insertion into a sphenoid sinus ostium, a Eustachian tube, and/or other anatomical passageways. Similarly, while the example described above correlates the second mode of operation and the first recess 234 with bending the guide rail 120 for insertion into the maxillary sinus ostium, the second mode of operation and the first recess 234 may also be used to bend the guide rail 120 for insertion into anatomical passageways other than the maxillary sinus ostium.

In some scenarios, it may be desirable to straighten a bent guide rail 120. By way of example only this may be accomplished using the bending tool 200 by positioning the bent guide rail 120 between the clamp member 250 and the block 230 while the clamp member 250 is spaced away from the block 230; then pressing the clamp member 250 toward the block 230. The resulting clamping of the bent guide rail 120 may straighten at least the region of the guide rail 120 that is clamped between the clamp member 250 and the block 230. To the extent that a region of the guide rail 120 that is not clamped between the clamp member 250 and the block 230 remains bent, the operator may grasp the bending tool 200 with one hand and grasp the instrument having the shaft assembly 100 with the other hand, and provide relative movement between the bending tool 200 and the instrument having the shaft assembly 100 to at least partially straighten a bent region of the guide rail 120 that is not clamped between the clamp member 250 and the block 230. In addition, or in the alternative, the operator may release the clamp member 250 to free the recently straightened region of the guide rail 120 from the space between the clamp member 250 and the block 230, then position a still-bent region of the guide rail 120 in the space between the clamp member 250 and the block 230, then press the clamp member 250 again toward the block 230 to straighten the otherwise still-bent region of the guide rail 120. As yet another variation, the bending tool 200 may include a tapered recess that is similar to a laterally extending recess 40 of the bending tool 10, which may be used to straighten a bent guide rail 120 as described above.

### C. Third Example of Bending Tool

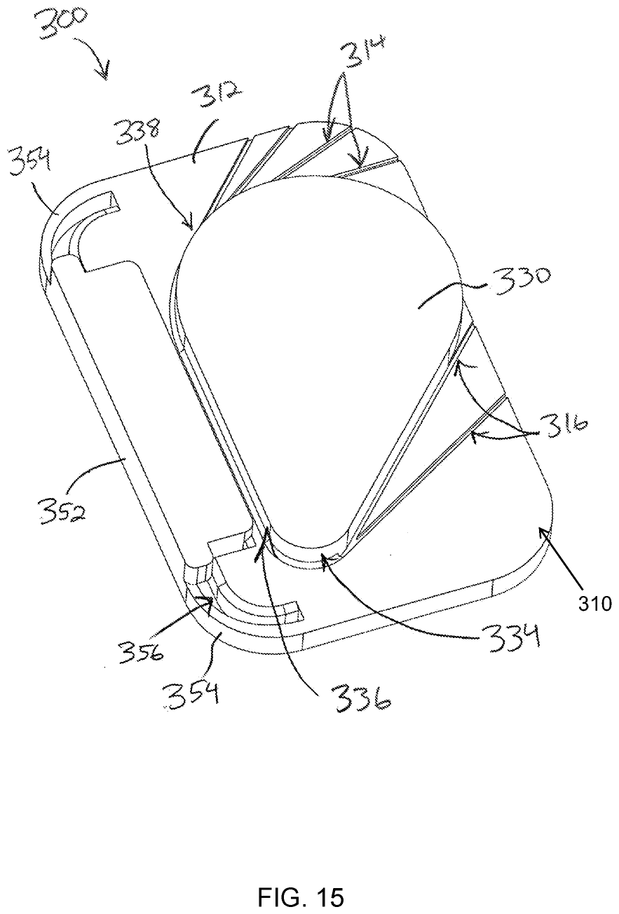

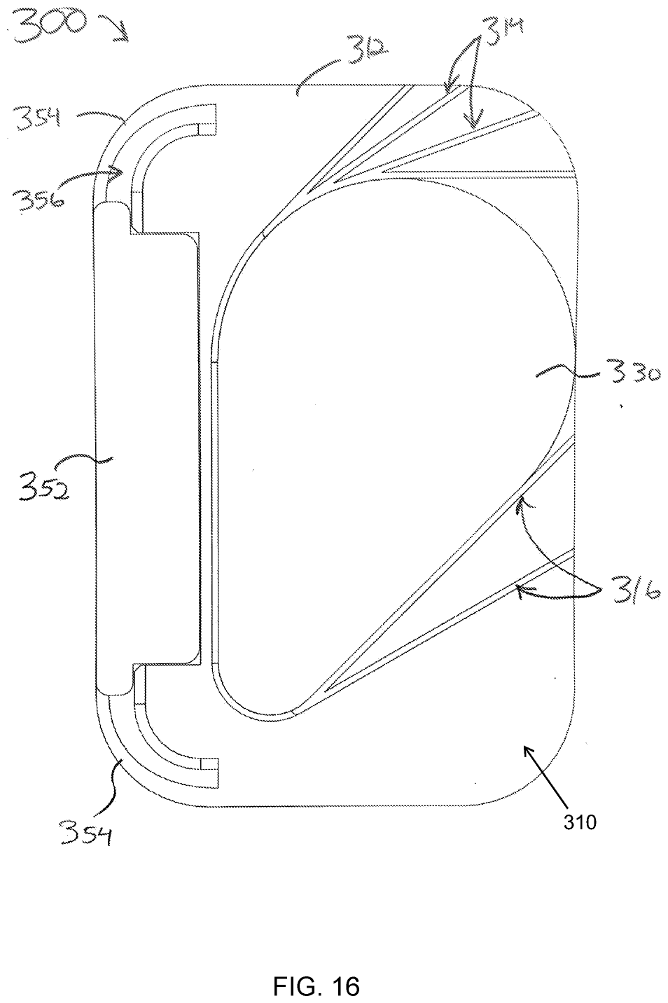

FIGS. 15-16 show another example of a bending tool 300 that may be used to bend a guide rail 120 of a shaft assembly 100. Except as otherwise described below, the bending tool 300 of this example may be configured and operable like the bending tool 200 described above. The bending tool 300 of this example includes a base 310 and a clamp member 352. The base 310 includes an upper surface 312 with a first set of indicia 314 and a second set of indicia 316, and an upwardly extending block 330. The clamp member 352 is secured to the base 310 by a pair of living hinges 354, which are configured to deform within gaps 354 formed between the living hinges 354 and the base 310. In the present example, the base 310, the clamp member 352, and the living hinges 354 are all formed as a single monolithic piece or a homogenous continuum of material. By way of example only, the bending tool 300 may be formed as a single piece of molded plastic. Alternatively, the bending tool 300 may be formed of any other suitable material(s) and using any other suitable technique(s).

The block 330 is configured to cooperate with the clamp member 352 to secure a distal portion of the guide rail 120 relative to the base 310. While not shown, the block 330 may include a set of windows that are configured and operable like the windows 232 described above. The block 330 of the present example includes a first radiused recess 334, a second radiused recess 336, and a third radiused recess 338. Each of the recesses 334, 336, 338 also has a radius along a first plane (e.g., vertical plane) that is configured to complement the radius of the guide rail 120, such that a laterally facing region of the guide rail 120 may fit in one or more of the recesses 334, 336, 338 during operation of the bending tool 300 as described in greater detail below. While each of the recesses 334, 336, 338 has the same radius of curvature extending inwardly into the block 330 along a first plane in this example, each of the recesses 334, 336, 338 is configured differently from the other recesses 334, 336, 338 around the perimeter of the block 330. In particular, the first recess 334 has a first radius of curvature along a second plane (e.g., horizontal plane), along a first region of the perimeter of the block 330. The second recess 336 extends along a straight path along a second region of the perimeter of the block 330, such that the second recess 336 does not have a radius of curvature along the second plane. The third recess 338 has a second radius of curvature along the second plane, along a third region of the perimeter of the block 330. The second radius of curvature is larger than the first radius of curvature. These differences in the radii of curvature of the recesses 334, 338 along the second plane may facilitate achievement of bend angles that are particularly suited for access by the guide rail 120 into different respective anatomical passageways, as described in greater detail below.

While not shown, the clamp member 352 may include a set of windows that are configured and operable like the windows 254 described above. As noted above, the clamp member 352 is secured to the base 310 by a pair of living hinges 354, which are configured to deform within the gaps 354 formed between the living hinges 354 and the base 310. The living hinges 354 are configured to resiliently urge the clamp member 352 away from the base 310, to thereby provide a space between the clamp member 352 and the block 330.

The bending tool 300 may be operable in two modes of operation like the two modes of operation described above with respect to the bending tool 200. For instance, in the orientation shown in FIG. 16, the bending tool 300 may be oriented for a mode of operation similar to the first mode of operation described above with respect to the bending tool 200 in the context of FIGS. 13A-13B. The operator may thus clamp the guide rail 120 between the clamp member 352 and the block 330, then bend the guide rail 120 along the third radiused recess 338 while observing the position of the shaft assembly 100 with respect to the indicia 314 until a desired bend angle is achieved. In the present example, this bend angle may be associated with access to the frontal recess, a sphenoid sinus ostium, a Eustachian tube, or some other anatomical passageway.

The operator may orient the bending tool 300 for use in a second mode of operation by rotating the bending tool 300 180 degrees along a horizontal plane, thereby reorienting the bending tool 300 to an orientation similar to that shown in FIG. 14 for the bending tool 200. In this second mode of operation, the operator may clamp the guide rail 120 between the clamp member 352 and the block 330, then bend the guide rail 120 along the third radiused recess 338 while observing the position of the shaft assembly 100 with respect to the indicia 316 until a desired bend angle is achieved. In the present example, this bend angle may be associated with access to the maxillary sinus ostium or some other anatomical passageway.

### D. Fourth Example of Bending Tool

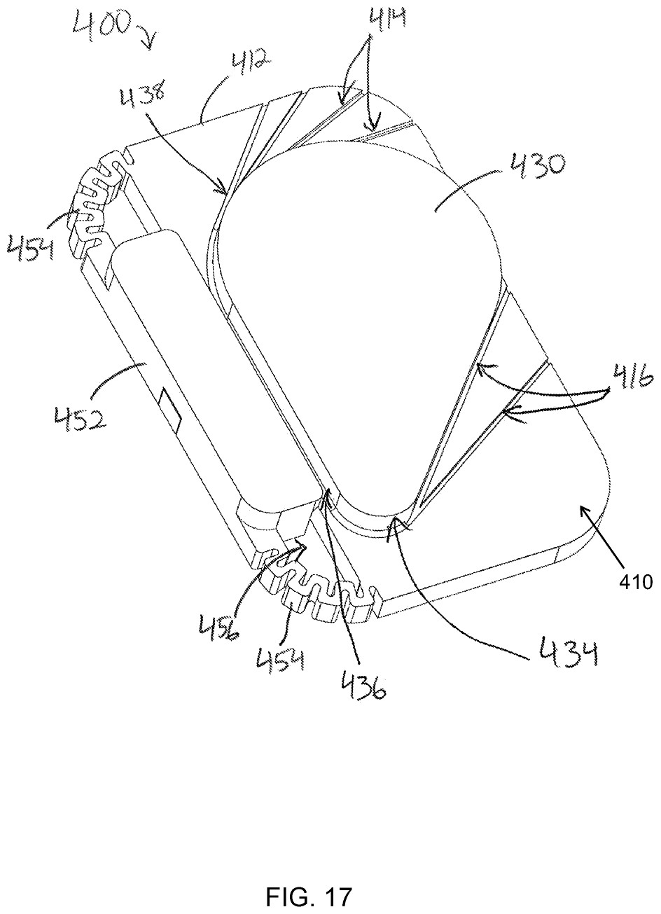

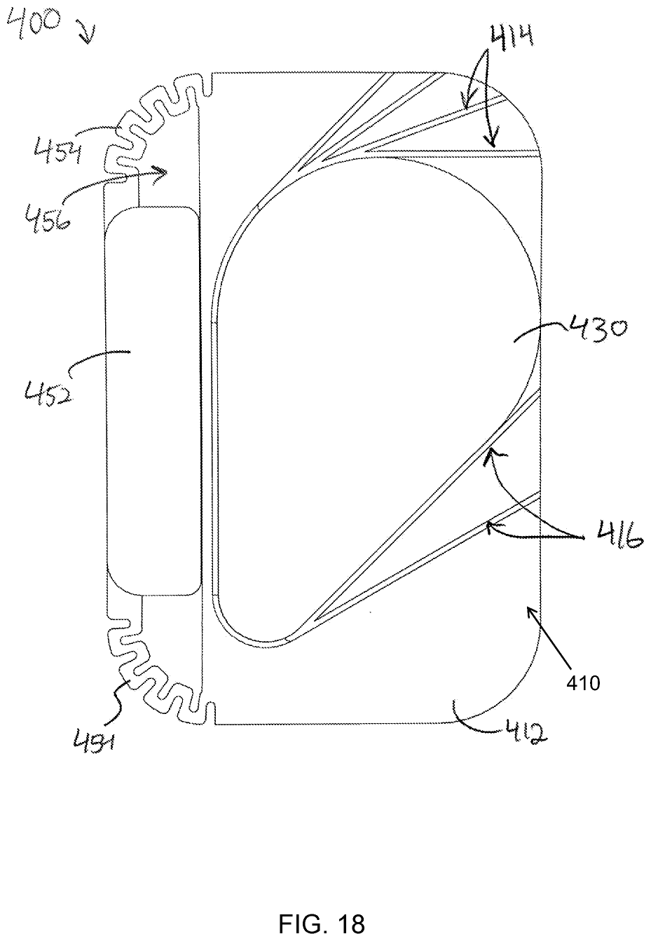

FIGS. 17-18 show another example of a bending tool 400 that may be used to bend a guide rail 120 of a shaft assembly 100. The bending tool 400 of this example is substantially identical to bending tool 300. The bending tool 400 thus includes a base 410 and a clamp member 452. The base 410 includes an upper surface 412 with a first set of indicia 414 and a second set of indicia 416, and an upwardly extending block 430. The clamp member 452 is secured to the base 410 by a pair of living hinges 454, which are configured to deform within gaps 454 formed between the living hinges 454 and the base 410. The block 430 includes a first radiused recess 434, a second radiused recess 436, and a third radiused recess 438. The recesses 434, 436, 438 are configured like the recesses 334, 336, 338 described above.

The bending tool 400 may be operated just like the bending tool 300 as described above. The only difference between the bending tool 400 and the bending tool 300 is that the living hinges 454 of the bending tool 400 have a serpentine configuration while the living hinges 354 of the bending tool 300 do not have a serpentine configuration. By way of example only, the serpentine configuration of the living hinges 454 may provide greater flexibility than the non-serpentine configuration of the living hinges 353.

### E. Fifth Example of Bending Tool

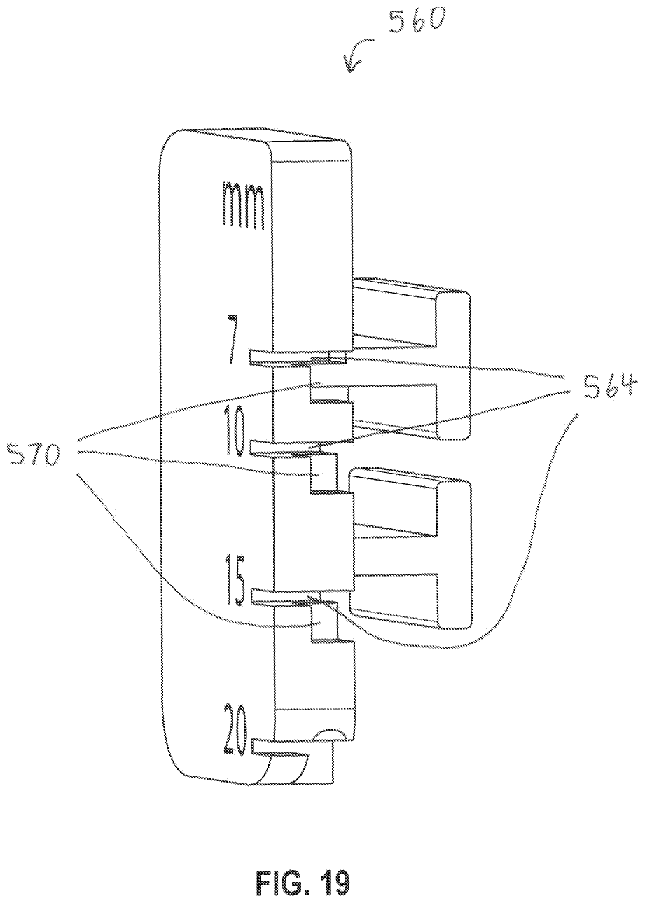

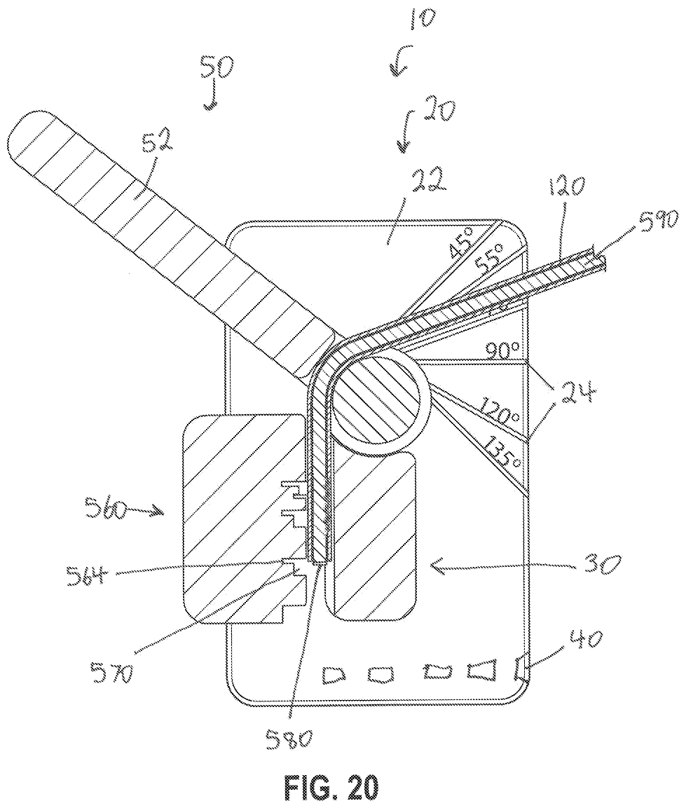

FIGS. 19-20 show another example of a clamp member 560 that is substantially similar to the clamp member 60 and insertable to the recesses 28 of the base 20. The clamp member 560 may function to secure the guide rail 120 against the block 30 in the same manner as the clamp member 60. The clamp member 560 of this example includes recesses 570 as shown in FIG. 19. In some versions, the clamp member 560 may also include a recess like the recess 66 of the clamp member 60. The recesses 570 are positioned to align with the windows 564 to allow clearance for an instrument having a sensor 580 mounted to a guide element 590 that is within the guide rail 120. As shown in FIG. 20, when the guide rail 120 is clamped between the clamp member 560 and the block 30, similar to FIG. 6C, the sensor 580 may be positioned within or adjacent to a recess 570 such that the sensor 580 remains undamaged. In other words, the recesses 570 may facilitate sufficient clamping of a guide rail 120 by the clamp member 560 without pinching the sensor 580 against the block 30 to a point where the sensor 580 is damaged by such pinching.

### F. Sixth Example of Bending Tool

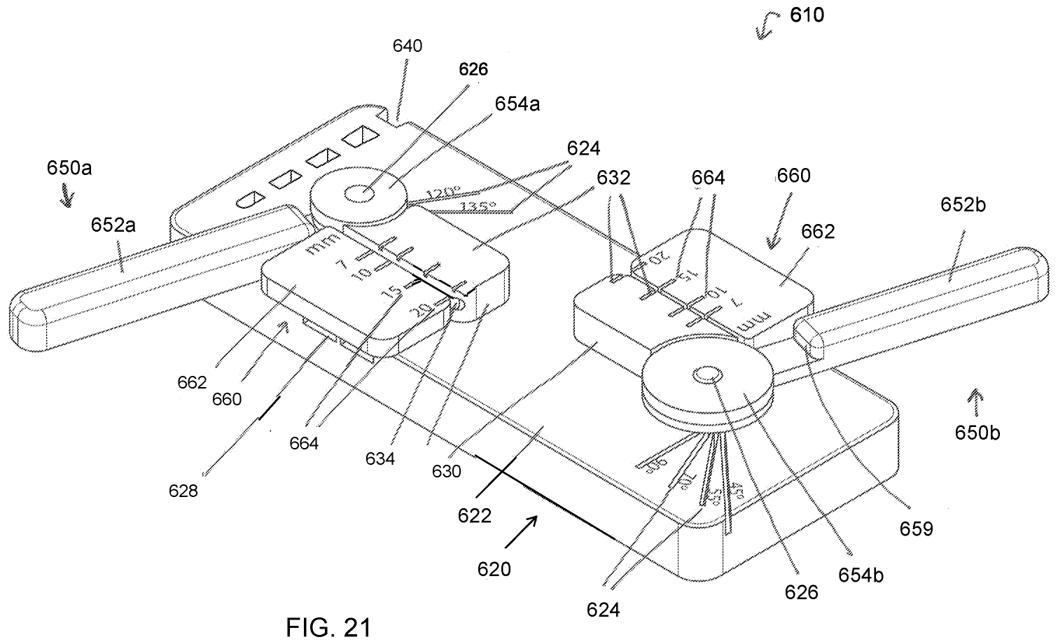

FIG. 21 shows another example of a bending tool 610 that may be used to bend the guide rail 120 of the shaft assembly 100. The bending tool 610 may be substantially similar to the bending tool 10 of FIG. 1 but with differences described below.

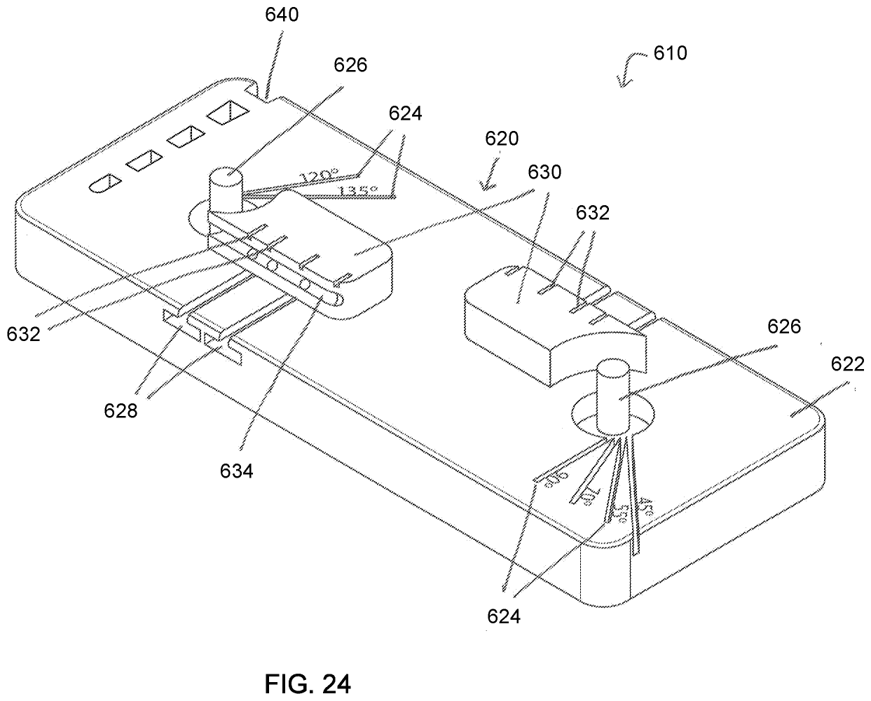

As shown in FIG. 21, the bending tool 610 of the present example comprises a base 620, actuators 650, and clamp members 660. As best seen in FIGS. 21 and 24, the base 620 includes an upper surface 622 with a series of indicia 624, upwardly extending posts 626, upwardly extending blocks 630, and a laterally extending recess 640. The indicia 624 are configured to visually indicate various bend angles as will be described in greater detail below. The posts 626 are configured to provide an axle about which a respective actuator 626 pivots relative to the base 620, as will also be described in greater detail below. The blocks 630 are configured to cooperate with a respective clamp member 60 to secure a distal portion of the guide rail 120 relative to the base 620, as will also be described in greater detail below. The blocks 630 each include a set of windows 632 and a radiused recess 634. The windows 632 are configured to provide indicia as described in greater detail below. The recess 634 also has a radius that is configured to complement the radius of the guide rail 120, such that a laterally facing region of the guide rail 120 may fit in the recess 634 during operation of the bending tool 610 as described in greater detail below. While the upwardly extending blocks 630, the upwardly extending posts 626, the actuators 650, and the clamp members 660 are shown on opposing ends of the upper surface 622 of the base 620, it is important to understand that any number of these components may be available on the base 620 and in any orientation. As an example, one set of an upwardly extending block 630, an upwardly extending post 626, an actuator 650, and a clamp member 660 may be on a lower surface (not shown) of the base 620 while another set may be on the upper surface 622 such that each of the posts 626 is in axial alignment with the other post 626, although axial alignment may not be necessary.

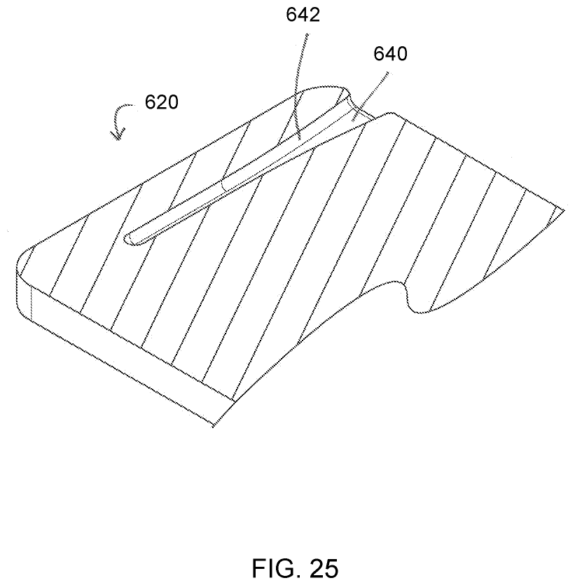

As best seen in FIG. 25, the recess 640 extends laterally into the side of the base 620, without passing fully through the base 620 in this version; and has a tapered surface 642 such that the opening of the recess 640 is wider than the furthest interior region of the recess 640. In the present example, the taper is curved, though in other versions, the taper may be angled or otherwise configured. In some other versions, the recess 640 passes fully through the entre width of the base 620. In some such versions, each open end of the recess 640 is wider than the interior region of the recess 640.

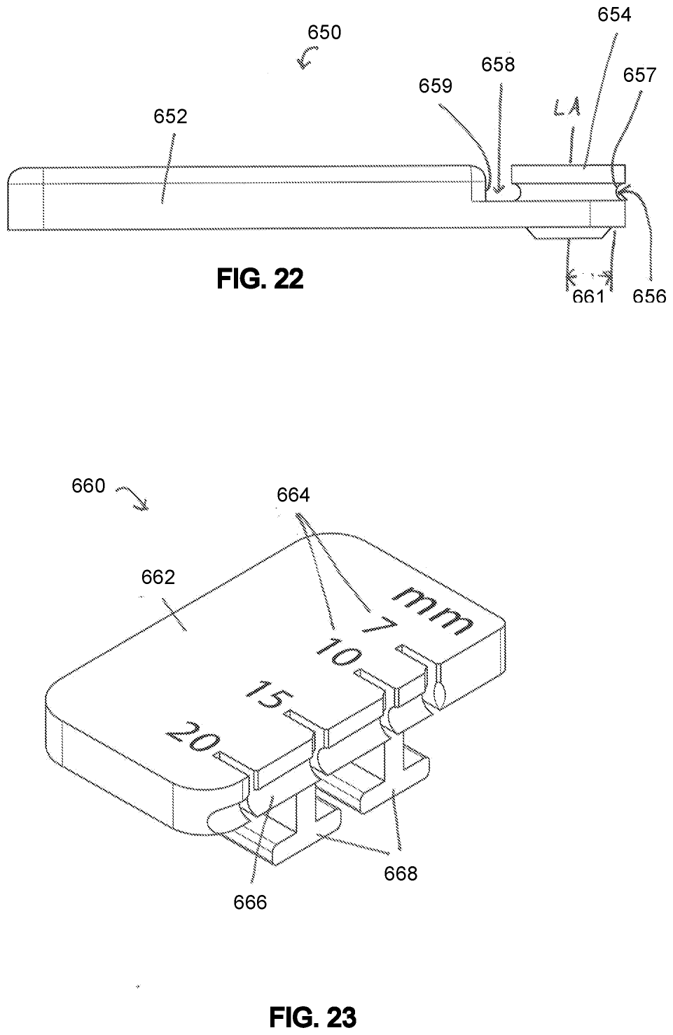

As shown in FIGS. 21-22, the actuators 650 each comprise a lever arm 652 and a hub 654. The hub 654 is pivotably secured to the base 620 via a respective post 626, such that the lever arm 652 is pivotable about a central axis defined by the respective post 626. As best seen in FIG. 22, each of the actuators 650 further includes a radiused annular recess 656 in the hub 654. In the present example, the annular recess 656 has a radius 657 that is configured to complement the radius of the guide rail 120, such that a laterally facing region of the guide rail 120 may fit in the annular recess 656 during operation of the bending tool 610 as described in greater detail below. Each of the annular recesses 656 of the various actuators 650 may include a bend radius 661 from a longitudinal axis (LA) of the hub 654 to the annular recess 656 that is different from any other of the annular recesses 656. For instance, one actuator 650b may include a larger bend radius 661 than another actuator 650a with a smaller bend radius 661. Including a different bend radius 661 on each of the hubs 654 allows an operator to bend a guide rail 120 at a desired radius. The indicia 624 may differ between the various sides of the bending tool 610 depending on which of the bend radii 661 is used. For instance, a larger bend radius 661 of the actuator 652b may be better suited for smaller bending angles (e.g., 45°, 55°, 70°, 90°, etc.); while a smaller bend radius 661 of the actuator 650a may be better suited for larger bending angles (e.g., 120°, 135°, etc.).

As best seen in FIGS. 21-22, a gap 658 is defined between the annular recess 656 and a bearing surface 659 of the lever arm 652. The gap 658 may differ across the various actuators 650 (as shown in FIG. 21) or they may be the same. Further radiused recess 634 of a respective upwardly extending block 630 may be spaced differently from a respective upwardly extending post 626 than another radiused recess 634 to accommodate for a different bend radius 661. This difference in spacing between the radiused recess 634 and the respective upwardly extending post 626 may be in any direction along a plane defined by the upper surface 622 of the base 620.

As shown in FIGS. 21 and 23, the clamp members 660 each comprise a body 662 having a set of windows 664, a radiused recess 666, and a pair of rails 668. The windows 664 are configured to correspond with the windows 632 and provide indicia as described in greater detail below. The rails 668 are configured to fit in complementary recesses 628 formed in the base 620, such that the clamp member 660 is slidably supported by the base 620. In the present example, the rails 668 and the recesses 628 have complementary T-shapes, though the rails 668 and the recesses 628 may alternatively have any other suitable shapes. The recess 666 has a radius that is configured to complement the radius of the guide rail 120, such that a laterally facing region of the guide rail 120 may fit in the recess 666 during operation of the bending tool 610 as described in greater detail below. The recess 666 of the clamp member 660 faces the recess 634 of the block 630, such that the guide rail 120 may be captured within the recesses 634, 666 during operation of the bending tool 610.

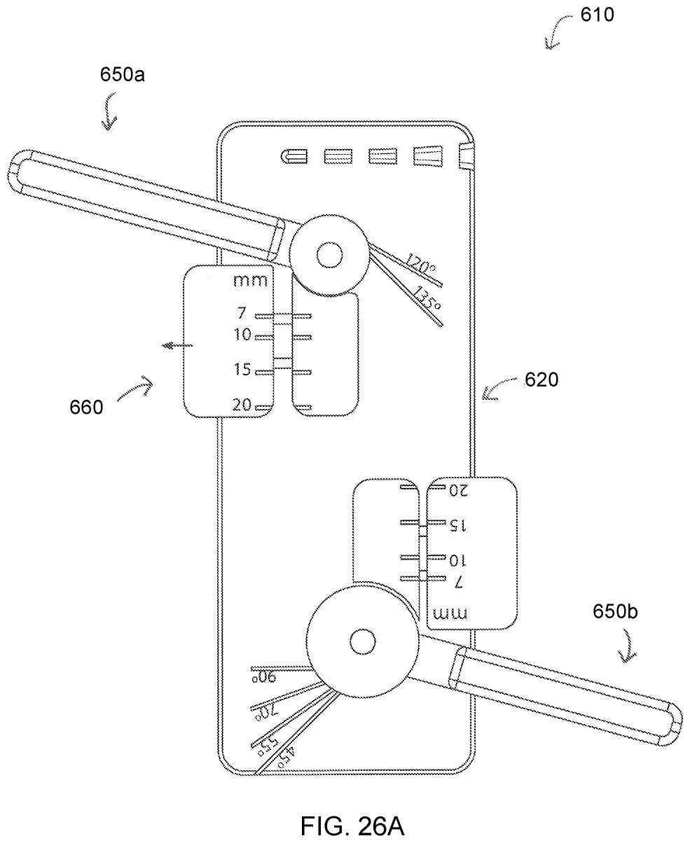

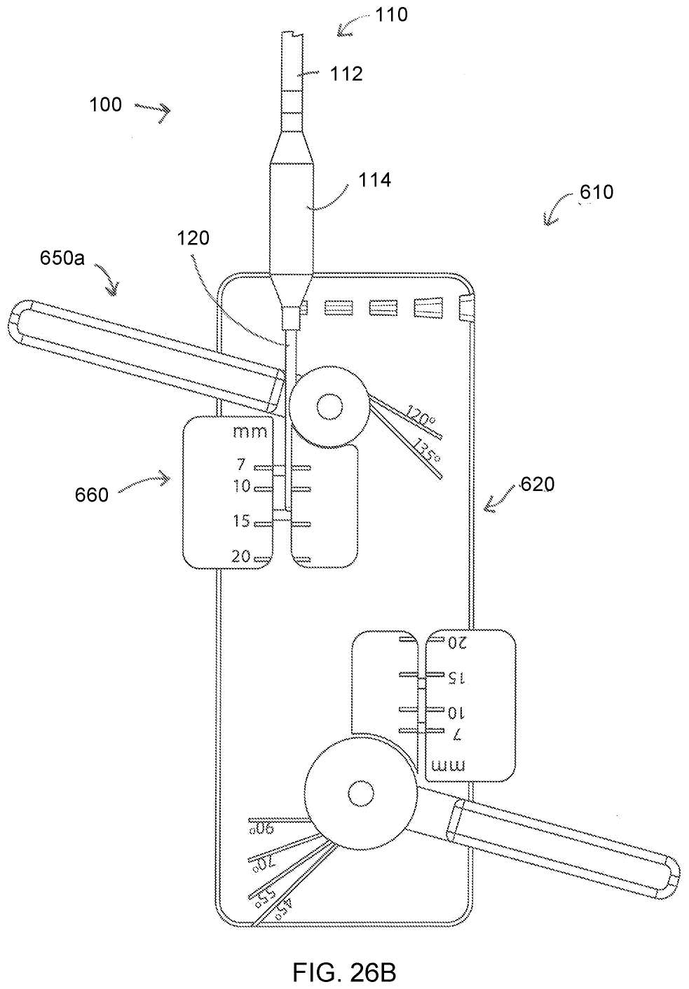

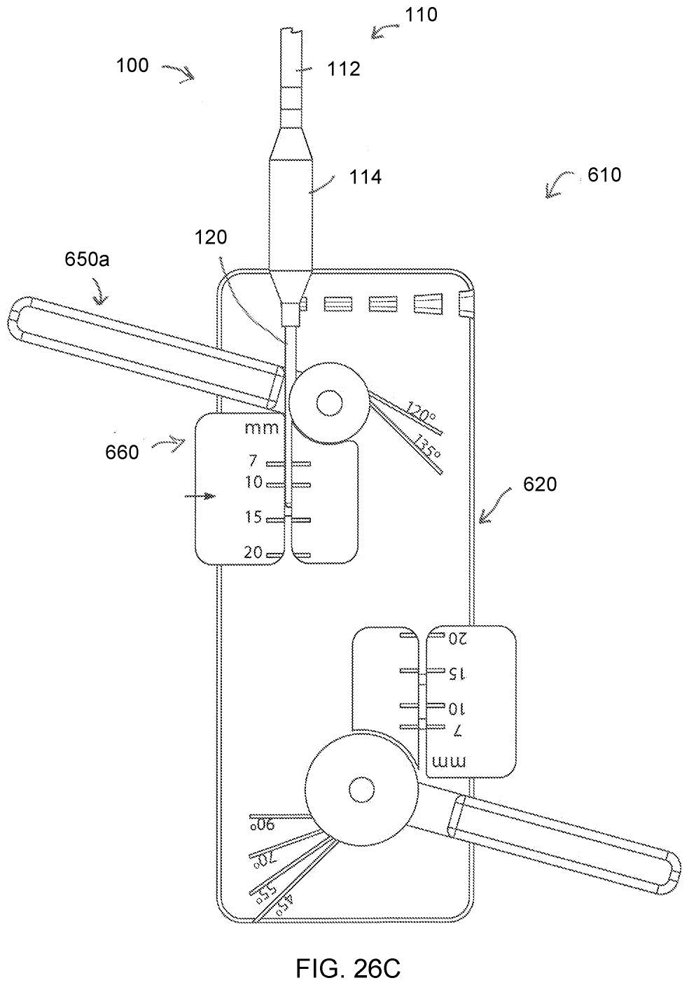

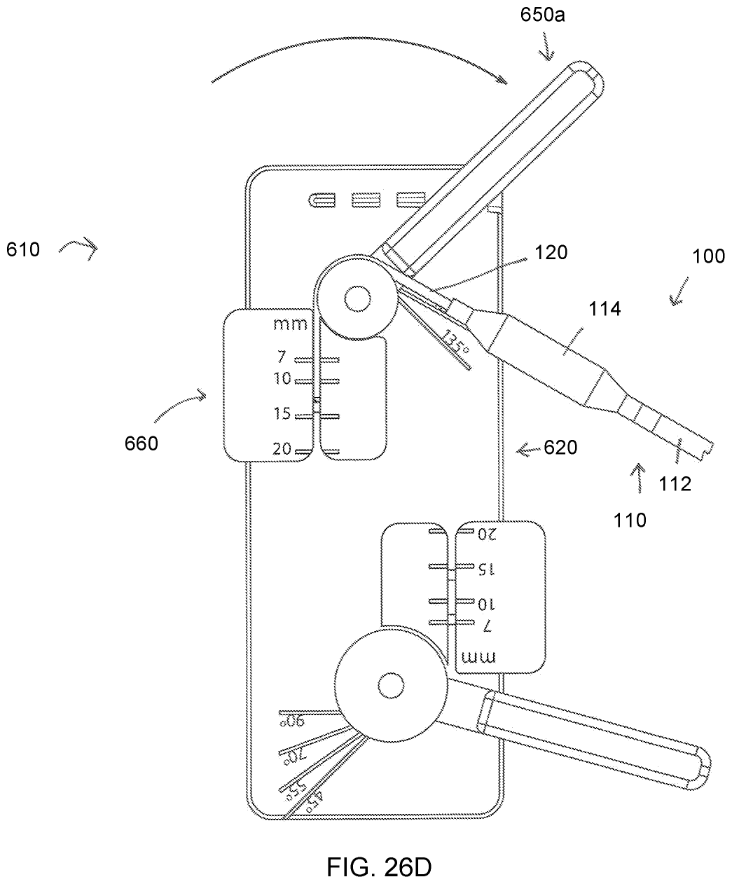

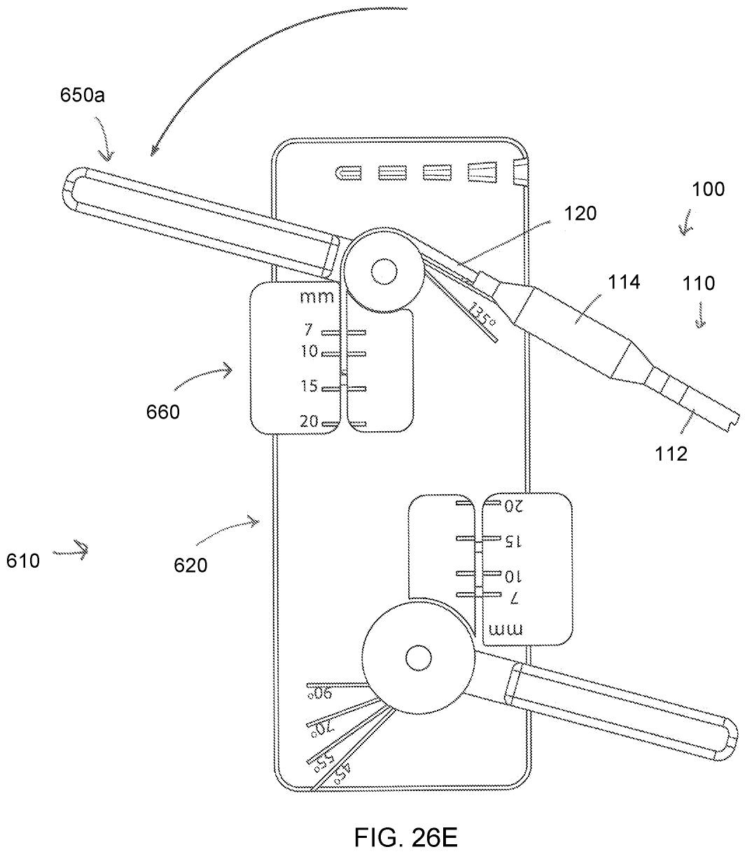

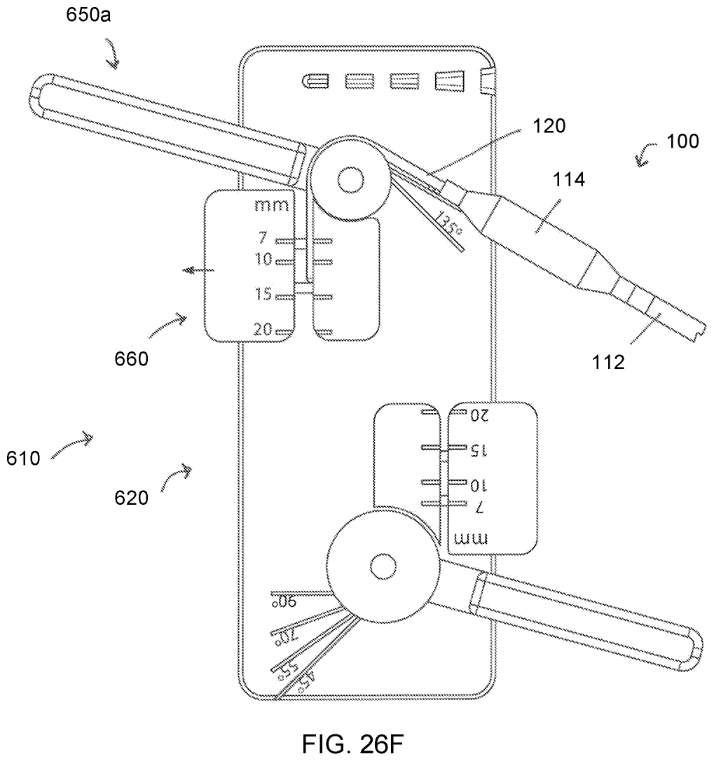

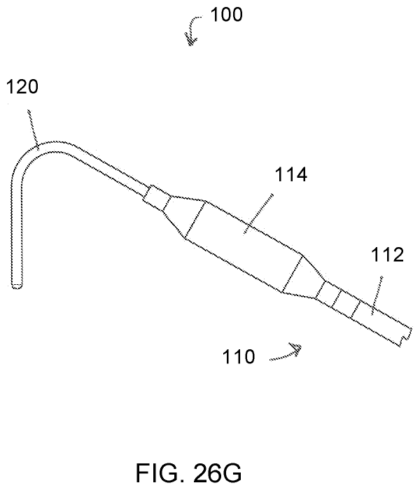

While each of the recesses 634, 666 has a semicircular, radiused profile in this example, either or both of the recesses 634, 666 may have any other suitable kind of profile, including but not limited to a V-shaped profile. In some variations, one of the recesses 634, 666 is omitted. Some variations of the bending tool 610 may also include detent features that provide tactile feedback and/or resistance against inadvertent movement of the clamp member 60 relative to the block 630. For instance, such detent features may provide tactile feedback and/or resistance against inadvertent movement of the clamp member 660 relative to the block 630 when the clamp member 660 is in a fully open position (e.g., as shown in FIGS. 26A-26B and 26F) and/or when the clamp member 660 is in a closed position (e.g., as shown in FIGS. 26C-26E). As shown in FIG. 21, the bending tool 610 may include a pair of clamp members 660 or may include one clamp member 660 that is interchangeable between the different sets of the recesses 628. The clamp members 660 may be substantially identical to one another or may differ in longitudinal length or an arrangement in the respective set of the windows 664 to thereby indicate different lengths. Further a single clamp member may be reversible such that a first set of the windows 664 appears on a first side of the clamp member 660 while a second set of the windows 664 appears on a second side of the clamp member 660 (not shown). The first and second sets of the windows 664 may differ in their distancing from a respective upper edge of the clamp member 660 which is positioned proximate a respective hub 654.

FIGS. 26A-26E show a particular side of the bending tool 610 being used to bend the guide rail 120 but it is important to understand that either side of the bending tool 610 may be used in the same manner as described below. As shown in FIG. 26A, operation of the bending tool 610 may select which bend radius 661 to achieve and then begin with the actuator 650 of the selected bend radius 661 pivoted fully counterclockwise; and with the clamp member 660 slid outward to maximize the gap between the clamp member 660 and the block 630. For purposes of the example described herein, the smaller bend radius 661 of the actuator 650a is used, however, the same process may be described for any alternative bend radius.

Next, as shown in FIG. 26B, the shaft assembly 100 may be positioned relative to the bending tool 610 such that the guide rail 120 is disposed in the gap 658; and such that the guide rail 120 is positioned to a desired depth between the clamp member 660 and the block 630. The depth of insertion of the guide rail 120 into the space between the clamp member 660 and the block 630 will determine the position of the bend angle along the guide rail 120. To facilitate achieving the desired depth of insertion, the operator may visually observe the position of the distal tip of the guide rail 120 in relation to the windows 632, 664. As shown, the clamp member 660 may further include depth markings for each of the windows 664. It should also be understood that the block 630 may include depth markings for each of the windows 632, in addition to or as an alternative to the clamp member 660 including depth markings for each of the windows 664. The depth of insertion (and thus the longitudinal position of the bend along the length of the guide rail 120) may be chosen based on the targeted anatomical structure, based on the anatomy of the patient at hand, and/or based on other considerations.

Once the shaft assembly 100 has been positioned relative to the bending tool 610 to achieve the desired depth of insertion of the guide rail 120 into the space between the clamp member 660 and the block 630, the operator may slide the clamp member 660 toward the block 630, to thereby capture the distal portion of the guide rail 120 in the recesses 634, 666, as shown in FIG. 26C. The configuration of the windows 632, 664 may allow the operator to visually confirm that the distal tip of the guide rail 120 is at the desired position after the distal portion of the guide rail 120 is captured in the recesses 634, 666. The operator may maintain a firm grip on the base 620 and the clamp member 660, to thereby firmly clamp the distal portion of the guide rail 120 between the clamp member 660 and the block 630. In some versions, the recesses 634, 666 include an elastomeric coating or overmold feature that further promotes gripping of the guide rail 120 between the clamp member 660 and the block 630.

With the distal portion of the guide rail 120 suitably clamped between the clamp member 660 and the block 630, the operator may then pivot the actuator 650 clockwise relative to the base 620 as shown in FIG. 26D. During this movement of the clamp member 660, the bearing surface 659 and the hub 654 may cooperate to form a bend in the guide rail 120. The operator may visually observe the guide rail 120 in relation to the indicia 624 to confirm when the desired bend angle has been achieved. After achieving the desired bend angle, the operator may pivot the actuator 650 counterclockwise relative to the base 620 as shown in FIG. 26E. The operator may then slide the clamp member 660 away from the block 630, to thereby release the distal portion of the guide rail 120, as shown in FIG. 26F. The operator may then remove the bending tool from the shaft assembly 100, leaving the guide rail 120 in a bent state with the formed bend having the desired bend angle at the desired longitudinal position along the length of the guide rail 120, as shown in FIG. 26G. After forming the bend with the desired bend angle at the desired longitudinal position along the length of the guide rail 120, the operator may use the shaft assembly 100 to dilate an anatomical passageway in the ear, nose, or throat of the patient; or otherwise use the shaft assembly 100.

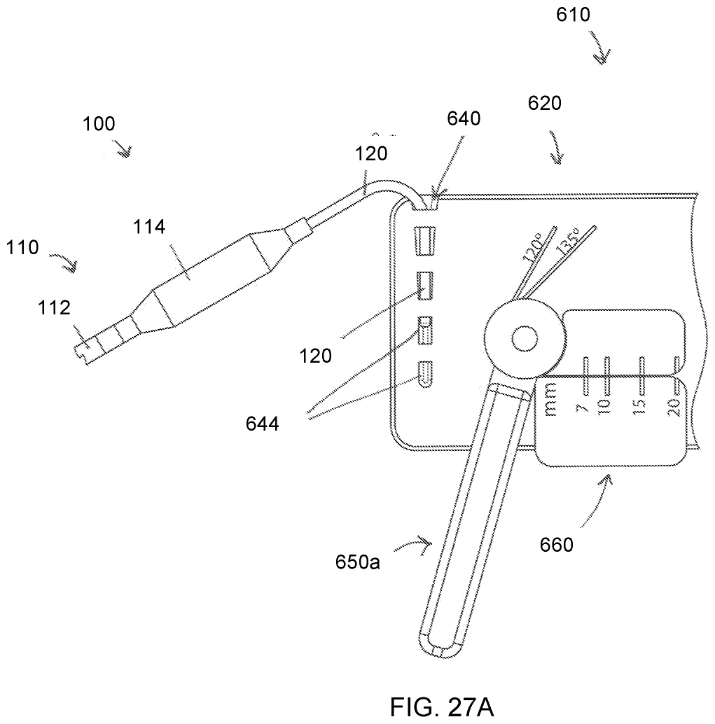

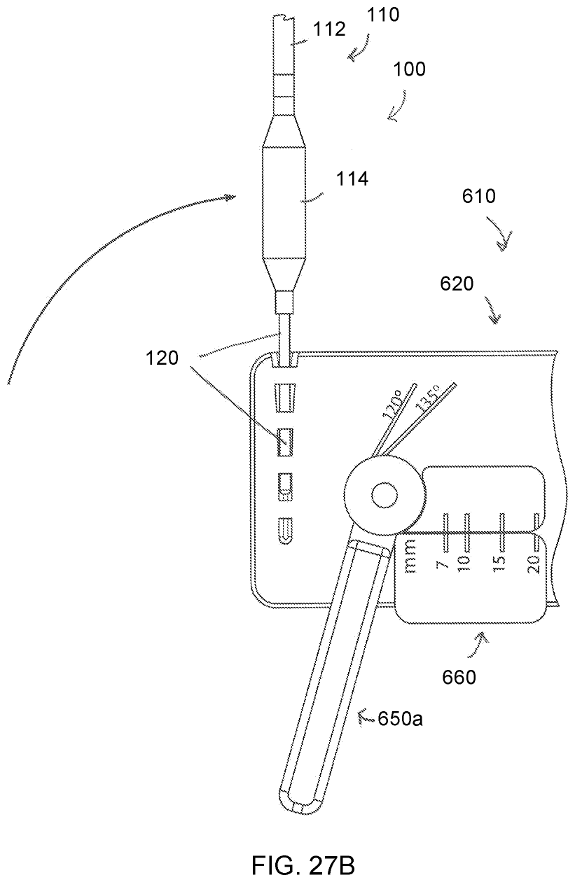

In some cases, the operator may wish to remove the bend that was formed in the guide rail 120. To that end, the operator may insert the bent guide rail 120 into the recess 640 of the base 620 as shown in FIG. 27A. In the present example, the base 620 includes a set of windows 644 formed through the upper surface 622, allowing the operator to visually observe the depth of insertion of the guide rail 120 into the recess 640. In some other versions, the windows 644 are omitted. With the guide rail 120 suitably disposed in the recess 640, the operator may grasp the base 620 with one hand and manipulate the shaft assembly 100 with the other hand to thereby bend the guide rail 120 back to a straightened state as shown in FIG. 27B. In some instances, the operator may re-bend the guide rail 120 to a new bend angle, perhaps at a different position along the length of the guide rail 120, using the procedure described above with reference to FIGS. 26A-26G. These bending and straightening processes may be repeated any desired number of times. In some scenarios, these processes are repeated to allow the shaft assembly 100 to be used to perform dilations in two or more different anatomical passageways (e.g., the maxillary sinus ostium, the frontal recess, the sphenoid sinus ostium, the Eustachian tube, etc.).

### II. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a base; (b) an actuator, the actuator being pivotably coupled with the base; and (c) a clamp member, the clamp member being operable to selectively clamp a malleable guide rail relative to the base, the guide rail being configured to fit in an anatomical passageway in a head of a patient, the actuator being operable to pivot relative to the base to thereby bend the guide rail while the guide rail is clamped by the clamp member.

### Example 2

The apparatus of Example 1, the base including a block, the clamp member being operable to clamp the guide rail against the block.

### Example 3

The apparatus of Example 2, the block including a recess, the recess being configured to receive a portion of the guide rail.

### Example 4

The apparatus of Example 3, the recess having a radiused profile.

### Example 5

The apparatus of any of Examples 2 through 4, the block further including indicia, the indicia being configured to indicate a depth of insertion of the guide rail into a space between the clamp member and the block.

### Example 6

The apparatus of any of Examples 2 through 5, the base defining an upper surface, the block extending upwardly from the upper surface.

### Example 7

The apparatus of any of Examples 1 through 6, the actuator including a lever arm and a hub, the actuator being pivotably coupled with the base via the hub.

### Example 8

The apparatus of Example 7, the base further including an integral post, the hub being pivotably coupled with the post.

### Example 9

The apparatus of any of Examples 7 through 8, the actuator further including a bearing surface, the actuator defining a gap between the hub and the bearing surface, the gap being sized to receive a portion of the guide rail, the bearing surface being configured to bear against the guide rail as the actuator is pivoted relative to the base.

### Example 10

The apparatus of any of Examples 7 through 9, the hub defining an annular recess, the annular recess being configured to receive a portion of the guide rail.

### Example 11

The apparatus of Example 10, the annular recess having a curved profile with a radius configured to complement a radius of the guide rail.

### Example 12

The apparatus of any of Examples 1 through 11, the base further including a set of indicia, the indicia being configured to visually indicate a plurality of bend angles.

### Example 13

The apparatus of any of Examples 1 through 12, the base further including a lateral recess, the lateral recess being configured to receive a bent region of the guide rail and provide straightening of the bent region of the guide rail.

### Example 14

The apparatus of Example 13, the lateral recess including an inner surface defining a taper.

### Example 15

The apparatus of any of Examples 13 through 14, the base further including a set of windows, the set of windows providing visibility into the lateral recess.

### Example 16

An apparatus, comprising: (a) a base, the base comprising a block; (b) an actuator, the actuator comprising a lever arm pivotably coupled with the base; and (c) a clamp member, the clamp member being slidably coupled with the base, the clamp member being operable to slide relative to the base between an open position and a closed position, the clamp member in the open position being configured to define a gap with the block, the gap being configured to allow positioning of a malleable guide rail between the clamp member and the block, the guide rail being configured to fit in an anatomical passageway in a head of a patient, the clamp member in the closed position being configured to clamp the malleable guide member against the block, the lever arm being configured to bend the malleable guide rail when the lever arm is pivoted relative to the base while the malleable guide member is clamped against the block.

### Example 17

The apparatus of Example 16, the clamp member and the block being configured to allow the malleable guide member to be positioned at a plurality of different longitudinal positions within the gap when the clamp member is in the open position, the clamp member and the block being configured to clamp the malleable guide member at any longitudinal position of the plurality of different longitudinal positions when the clamp member is in the closed position.

### Example 18

The apparatus of any of Examples 16 through 17, the apparatus being configured to allow an operator to form a bend in the malleable guide rail at any selected position along the length of the malleable guide rail within a range from approximately 20 mm from the distal tip of the guide rail to approximately 7 mm from the distal tip of the guide rail.

### Example 19

The apparatus of any of Examples 16 through 18, the base including indicia configured to enable an operator to visually confirm formation of a bend in the malleable guide rail at any selected bend angle within a range from approximately 45 degrees to approximately 135 degrees.

### Example 20

A method, comprising: (a) positioning a malleable guide rail at a selected longitudinal position within a gap defined between a clamp member of a bending tool and a block of the bending tool; (b) moving the clamp member toward the block to thereby clamp the malleable guide rail against the block at the selected longitudinal position; and (c) pivoting an actuator of the bending tool relative to a base of the bending tool, while the guide rail is clamped against the block by the clamp member, to achieve a desired bend angle in the guide rail, the guide rail being configured to fit in an anatomical passageway in a head of a patient.

### Example 21

An apparatus, comprising: (a) a base, the base including a block, the block defining an outer perimeter extending along a first plane, the outer perimeter including a bending surface having a first region, a second region, and a third region, the second region being interposed between the first region and the second region along the outer perimeter, the first region extending along a curved path having a first radius of curvature along the first plane, the second region extending along a straight path along the first plane, the third region extending along a curved path having a second radius of curvature; (b) a clamp member, the clamp member being operable to clamp a first portion of a malleable guide rail against the second region of the bending surface; the first region of the bending surface being operable to bend a second portion of the malleable guide rail along the curved path having the first radius of curvature while the first region of the malleable guide rail is clamped between the clamp member and the second region of the bending surface in a first mode of operation; the third region of the bending surface being operable to bend the second portion of the malleable guide rail along the curved path having the first radius of curvature while the first region of the malleable guide rail is clamped between the clamp member and the second region of the bending surface in a second mode of operation.

### Example 22

The apparatus of Example 21, the bending surface being curved along a second plane that is perpendicular to the first plane.

### Example 23

The apparatus of Example 22, the curvature of the bending surface along the second plane being configured to complement a curvature of an outer diameter of the malleable guide rail.

### Example 24

The apparatus of any of Examples 21 through 23, the block further including indicia, the indicia being configured to indicate a depth of insertion of the guide rail into a space between the clamp member and the block.

### Example 25

The apparatus of any of Examples 21 through 24, the clamp member further including indicia, the indicia being configured to indicate a depth of insertion of the guide rail into a space between the clamp member and the block.

### Example 26

The apparatus of any of Examples 21 through 25, the base defining an upper surface, the block extending upwardly from the upper surface.

### Example 27

The apparatus of Example 26, the base further including indicia on the upper surface, the indicia being configured to indicate a bend angle of a guide rail clamped between the clamp member and the block.

### Example 28

The apparatus of Example 27, the indicia on the upper surface including a first set of indicia and a second set of indicia, the first set of indicia being configured to indicate a bend angle of a guide rail clamped between the clamp member and the block during the first mode of operation, the second set of indicia being configured to indicate a bend angle of a guide rail clamped between the clamp member and the block during the second mode of operation.

### Example 29

The apparatus of any of Examples 21 through 28, the clamp member being resiliently biased away from the base.

### Example 30

The apparatus of Example 29, further comprising a spring interposed between the clamp member and the base to resiliently bias the clamp member away from the base.

### Example 31

The apparatus of Example 29, further comprising a pair of living hinges coupling the clamp member with the base, the pair of living hinges being configured to resiliently bias the clamp member away from the base.

### Example 32

The apparatus of Example 31, each living hinge of the pair of living hinges having a serpentine configuration.

### Example 33

A method, comprising: (a) positioning a malleable guide rail at a selected longitudinal position within a gap defined between a clamp member of a bending tool and a block of the bending tool; (b) moving the clamp member toward the block to thereby clamp the guide rail against a bending surface of the block at the selected longitudinal position, the bending surface extending along a first plane, the bending surface having a first region, a second region, and a third region, the second region being interposed between the first region and the second region, the first region extending along a curved path having a first radius of curvature along the first plane, the second region extending along a straight path along the first plane, the third region extending along a curved path having a second radius of curvature, the guide rail being clamped against the second region; and (c) bending the guide rail against a selected one of the first region of the bending surface or the second region of the bending surface, while the guide rail is clamped against the block by the clamp member, to achieve a desired bend angle in the guide rail, the guide rail being configured to fit in an anatomical passageway in a head of a patient.

### Example 34

The method of Example 33, the act of positioning the guide rail comprising inserting the guide rail along a first insertion direction into the gap defined between the clamp member and the block, the act of bending the guide rail comprising bending the guide rail along the first region of the bending surface, thereby providing a bend in the guide rail with the first radius of curvature.

### Example 35

The method of Example 34, further comprising: (a) inserting the guide rail along a second insertion direction into the gap defined between the clamp member and the block, the second insertion direction being opposite to the first insertion direction; and (b) bending the guide rail along the third region of the bending surface, thereby providing a bend in the guide rail with the second radius of curvature.

### Example 36

The method of Example 35, further comprising rotating the bending tool approximately 180 degrees along the first plane between the act of bending the guide rail along the first region of the bending surface and inserting the guide rail along the second insertion direction into the gap defined between the clamp member and the block.

### Example 37

The method of any of Examples 35 through 36, further comprising at least partially straightening the bent guide rail between the act of bending the guide rail along the first region of the bending surface and inserting the guide rail along the second insertion direction into the gap defined between the clamp member and the block.

### Example 38

The method of any of Examples 35 through 37, further comprising: (a) observing a position of the guide rail relative to a first set of indicia on the bending tool while bending the guide rail along the first region of the bending surface; and (b) observing a position of the guide rail relative to a second set of indicia on the bending tool while bending the guide rail along the second region of the bending surface.

### Example 39

The method of any of Examples 35 through 38, the act of bending the guide rail along the first region of the bending surface providing a bend angle in the guide rail that is up to approximately 90 degrees, the act of bending the guide rail along the first region of the bending surface providing a bend angle in the guide rail that is at least approximately 120 degrees.

### Example 40

The method of any of Examples 33 through 39, further comprising inserting the bent guide rail into an anatomical passageway within a head of a patient.

### Example 41

An apparatus, comprising: (a) a base; (b) a plurality of actuators, each actuator of the plurality of actuators being pivotably coupled with the base and including a bend radius, each bend radius of a respective actuator being different from any other bend radius of a respective actuator of the plurality of actuators; and (c) a clamp member, the clamp member being operable to selectively clamp a malleable guide rail relative to the base, the guide rail being configured to fit in an anatomical passageway in a head of a patient, each actuator of the plurality of actuators being operable to pivot relative to the base to thereby bend the guide rail along a respective bend radius while the guide rail is clamped by the clamp member.

### Example 42

The apparatus of Example 41, the base including a block, the clamp member being operable to clamp the guide rail against the block.

### Example 43

The apparatus of Example 42, the block including a recess, the recess being configured to receive a portion of the guide rail.

### Example 44

The apparatus of Example 43, the recess having a radiused profile.

### Example 45

The apparatus of any of Examples 42 through 44, the block further including indicia, the indicia being configured to indicate a depth of insertion of the guide rail into a space between the clamp member and the block.

### Example 46

The apparatus of any of Examples 42 through 45, the base defining an upper surface, the block extending upwardly from the upper surface.

### Example 47

The apparatus of any of Examples 41 through 46, each actuator of the plurality of actuators including a lever arm and a hub, each actuator of the plurality of actuators being pivotably coupled with the base via the hub, the hub of each actuator providing the bend radius of the corresponding actuator.

### Example 48

The apparatus of Example 47, the base further including a plurality of posts, each hub being pivotably coupled with a respective post of the plurality of posts.

### Example 49

The apparatus of any of Examples 47 through 48, each actuator of the plurality of actuators further including a bearing surface, each actuator of the plurality of actuators defining a respective gap between the respective hub and the respective bearing surface, the respective gap being sized to receive a portion of the guide rail, the respective bearing surface being configured to bear against the guide rail as the actuator is pivoted relative to the base, the respective gap of an actuator being different than a gap of any other actuator of the plurality of actuators.

### Example 50

The apparatus of any of Examples 47 through 49, the hub of each actuator of the plurality of actuators defining an annular recess, each annular recess being configured to receive a portion of the guide rail.

### Example 51

The apparatus of Example 50, each annular recess having a curved profile with a radius configured to complement a radius of the guide rail.

### Example 52

The apparatus of any of Examples 41 through 51, the base further including a set of indicia, the indicia being configured to visually indicate a plurality of bend angles.

### Example 53

The apparatus of any of Examples 41 through 52, the base further including a lateral recess, the lateral recess being configured to receive a bent region of the guide rail and provide straightening of the bent region of the guide rail.

### Example 54

The apparatus of Example 53, the lateral recess including an inner surface defining a taper.

### Example 55

The apparatus of any of Examples 53 through 54, the base further including a set of windows, the set of windows providing visibility into the lateral recess.

### Example 56

An apparatus, comprising: (a) a base, the base comprising a block; (b) a plurality of actuators, each actuator of the plurality of actuators comprising a lever arm pivotably coupled with the base and including a bend radius, each bend radius of a respective actuator being different from any other bend radius of a respective actuator of the plurality of actuators; and (c) a clamp member, the clamp member being slidably coupled with the base, the clamp member being operable to slide relative to the base between an open position and a closed position, the clamp member in the open position being configured to define a gap with the block, the gap being configured to allow positioning of a malleable guide rail between the clamp member and the block, the guide rail being configured to fit in an anatomical passageway in a head of a patient, the clamp member in the closed position being configured to clamp the malleable guide member against the block, the lever arm of a respective actuator being configured to bend the malleable guide rail along the respective bend radius when the lever arm is pivoted relative to the base while the malleable guide member is clamped against the block.

### Example 57

The apparatus of Example 56, the clamp member and the block being configured to allow the malleable guide member to be positioned at a plurality of different longitudinal positions within the gap when the clamp member is in the open position, the clamp member and the block being configured to clamp the malleable guide member at any longitudinal position of the plurality of different longitudinal positions when the clamp member is in the closed position.

### Example 58

The apparatus of any of Examples 56 through 57, the apparatus being configured to allow an operator to form a bend in the malleable guide rail at any selected position along the length of the malleable guide rail within a range from approximately 20 mm from the distal tip of the guide rail to approximately 7 mm from the distal tip of the guide rail.

### Example 59

The apparatus of any of Examples 56 through 58, the base including indicia configured to enable an operator to visually confirm formation of a bend in the malleable guide rail at any selected bend angle within a range from approximately 45 degrees to approximately 135 degrees.

### Example 60

A method, comprising: (a) selecting a bend radius between a first bend radius and a second bend radius, the first bend radius being different than the second bend radius; (b) positioning a malleable guide rail at a selected longitudinal position within a gap defined between a clamp member of a bending tool and a block of the bending tool; (c) moving the clamp member toward the block to thereby clamp the malleable guide rail against the block at the selected longitudinal position; and (d) pivoting an actuator of the bending tool relative to a base of the bending tool, while the guide rail is clamped against the block by the clamp member, to achieve a desired bend angle having the selected bend radius in the guide rail, the guide rail being configured to fit in an anatomical passageway in a head of a patient.

### III. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus, comprising:
a base;
an actuator, the actuator being pivotably coupled with the base; and
a clamp member, the clamp member being operable to selectively clamp a malleable guide rail relative to the base, the guide rail being configured to fit in an anatomical passageway in a head of a patient, the actuator being operable to pivot relative to the base to thereby bend the guide rail while the guide rail is clamped by the clamp member.

2. The apparatus of claim 1, wherein:
the base includes a block, and
the clamp member is operable to clamp the guide rail against the block.

3. The apparatus of claim 2, wherein:
the block includes a recess, and
the recess is configured to receive a portion of the guide rail.

4. The apparatus of claim 3, wherein the recess has a radiused profile.

5. The apparatus of claim 2, wherein:
the block further includes indicia, and
the indicia is configured to indicate a depth of insertion of the guide rail into a space between the clamp member and the block.

6. The apparatus of claim 2, wherein:
the base defines an upper surface, and
the block extends upwardly from the upper surface.

7. The apparatus of claim 1, wherein:
the actuator includes a lever arm and a hub, and
the actuator is pivotably coupled with the base via the hub.

8. The apparatus of claim 7, wherein:
the base further includes an integral post, and
the hub is pivotably coupled with the post.

9. The apparatus of claim 7, wherein:
the actuator further includes a bearing surface,
the actuator defines a gap between the hub and the bearing surface, the gap being sized to receive a portion of the guide rail, and
the bearing surface is configured to bear against the guide rail as the actuator is pivoted relative to the base.

10. The apparatus of claim 7, wherein:
the hub defines an annular recess, and
the annular recess is configured to receive a portion of the guide rail.

11. The apparatus of claim 10, wherein the annular recess has a curved profile with a radius configured to complement a radius of the guide rail.

12. The apparatus of claim 1, wherein the base further includes a set of indicia, the indicia being configured to visually indicate a plurality of bend angles.

13. The apparatus of claim 1, wherein the base further includes a lateral recess, the lateral recess is configured to receive a bent region of the guide rail and provide straightening of the bent region of the guide rail.

14. The apparatus of claim 13, wherein the lateral recess includes an inner surface defining a taper.

15. The apparatus of claim 13, wherein the base further includes a set of windows, the set of windows providing visibility into the lateral recess.

16. An apparatus, comprising:
a base, the base comprising a block;
an actuator, the actuator comprising a lever arm pivotably coupled with the base; and
a clamp member, the clamp member being slidably coupled with the base, the clamp member being operable to slide relative to the base between an open position and a closed position,
wherein (i) the clamp member in the open position being configured to define a gap with the block, the gap being configured to allow positioning of a malleable guide rail between the clamp member and the block, the guide rail being configured to fit in an anatomical passageway in a head of a patient, (ii) the clamp member in the closed position being configured to clamp the malleable guide member against the block, and (iii) the lever arm being configured to bend the malleable guide rail when the lever arm is pivoted relative to the base while the malleable guide member is clamped against the block.

17. The apparatus of claim 16, wherein the clamp member and the block are configured to:
allow the malleable guide member to be positioned at a plurality of different longitudinal positions within the gap when the clamp member is in the open position, and
clamp the malleable guide member at any longitudinal position of the plurality of different longitudinal positions when the clamp member is in the closed position.

18. The apparatus of claim 16, wherein the apparatus is configured to allow an operator to form a bend in the malleable guide rail at any selected position along the length of the malleable guide rail within a range from approximately 20 mm from the distal tip of the guide rail to approximately 7 mm from the distal tip of the guide rail.

19. The apparatus of claim 16, wherein the base includes indicia configured to enable an operator to visually confirm formation of a bend in the malleable guide rail at any selected bend angle within a range from approximately 45 degrees to approximately 135 degrees.

20. A method, comprising:
positioning a malleable guide rail at a selected longitudinal position within a gap defined between a clamp member of a bending tool and a block of the bending tool;
moving the clamp member toward the block to thereby clamp the malleable guide rail against the block at the selected longitudinal position; and
pivoting an actuator of the bending tool relative to a base of the bending tool, while the guide rail is clamped against the block by the clamp member, to achieve a desired bend angle in the guide rail, the guide rail being configured to fit in an anatomical passageway in a head of a patient.
